(19) United States
(12) Patent Application Publication
(10) Pub. No.: US 2025/0197531 A1
(43) Pub. Date: Jun. 19, 2025

(54) COMPOSITION, IN THE FORM OF AN AQUEOUS SOLUTION COMPRISING AT LEAST ONE MACROMOLECULAR COMPOUND

(71) Applicant: LABORATOIRES VIVACY, PARIS (FR)

(72) Inventors: Frederic TRANCHEPAIN, VILLY LE BOUVERET (FR); Florence BRUNEL, ANNEMASSE (FR)

(73) Assignee: LABORATOIRES VIVACY, PARIS (FR)

(21) Appl. No.: 18/707,619

(22) PCT Filed: Nov. 7, 2022
(86) PCT No.: PCT/EP2022/081024

§ 371 Date: Feb. 24, 2025

(30) Foreign Application Priority Data
Nov. 6, 2021 (EP) 21206821.7

(51) Int. Cl. C08B 37/00

(52) U.S. Cl. CPC C08B 37/00

(57) ABSTRACT

A composition, in the form of an aqueous solution, filterable on a membrane with a porosity of 0.22 μm including at least one macromolecular compound consisting of sequences of identical or different polysaccharides linked together by a divalent radical L. L is a divalent radical resulting from a reaction between a crosslinking agent and two reactive functions, each reactive function being carried by two distinct polysaccharide chains and the chains of polysaccharides do not form cyclic structures.

The Invention relates to the field of polysaccharide-based formulations used as biomaterials, more particularly in the medical and aesthetic fields. In these applications, the formulations must have optimised properties in terms of rheology taking into account in particular the characteristics of the medical device used for the injection, the characteristics of the treated area and the requisite therapeutic or cosmetic effect.

Products in the form of gel based on crosslinked polysaccharide used for aesthetic application have rheological properties optimised to have good stability in the injection area, The gels used have in particular a firmness to deformation which results in a loss tangent, Tan Δ (Tn δ), less than 1.00, the elastic modulus G′ being much greater than the viscosity modulus G″ on a wide deformation range. In particular, most products used in gel form have a loss tangent of less than 0.50 (REF). This characteristic of gel-based products imposes a certain constraint in terms of injectability, which can limit their range of use. In particular, using such a product for sensitive areas of the body or face can be delicate, or even problematic.

The present invention relates to the development of compositions in the form of aqueous solutions, having very good fluidity, filterable on a membrane with a porosity of 0.22 μm, these compositions comprising at least one linear or branched macromolecular compound consisting of a sequence of polysaccharides.

In patent application WO2020250128, filed by the company OFFHEALTH SPA, filterable compositions are disclosed on membranes with a porosity of 0.2 μm comprising nanoparticles of crosslinked hyaluronic acid obtained by the mechanical action of the stirring system on crosslinked hyaluronic acid. These nanoparticles are suspended in the compositions disclosed. Consequently, these compositions do not contain macromolecules equivalent to those obtained by the process of the present invention and no rheological property of the compositions is evaluated because they are compositions comprising nanoparticles in suspension and not viscoelastic compositions.

In patent application WO2016030516, filed by the company GALDERMA SA, the possibility of filtering on membranes with a porosity of 0.2 μm, the supernatant resulting from the thermal, enzymatic or radical degradation of gels containing hyaluronic acid is presented. Although these compositions are filterable on membranes with a porosity of 0.2 μm, the rheological properties of these filtrates are not comparable to those of the compositions disclosed in the present invention and they are not suitable for the applications referred to in the present application.

Surprisingly, the compositions according to the invention have the remarkable advantage of being filterable on a membrane with a porosity of 0.22 μm, which makes it possible to incorporate thermosensitive compounds into them such as peptides, proteins, growth factors, antibodies or vitamins in particular, because the compositions obtained can then be sterilised by simple filtration. Existing products based on crosslinked polysaccharide gels are incompatible with heat-sensitive compounds because autoclave treatment or heat sterilization of the gel is necessary to avoid any risk of infection on the area treated by injection.

The present invention also relates to compositions comprising at least one macromolecular compound consisting of a sequence of hyaluronic acids having, after injection, good durability and resistance to enzymatic degradation, in particular.

The compositions according to the present invention also allow the preparation of solutions with high polysaccharide concentrations, in particular hyaluronic acid, while remaining easily injectable.

The present invention relates to a composition, in the form of an aqueous solution, filterable on a membrane with a porosity of 0.22 μm comprising at least one macromolecular compound consisting of sequences of identical or different polysaccharides linked together by a divalent radical L.

L is a divalent radical resulting from a reaction between a crosslinking agent and two reactive functions, each reactive function being carried by two distinct polysaccharide chains and the chains of polysaccharides do not form cyclic structures.

In one embodiment, the composition according to the invention is characterised in that the macromolecular compound consists of a sequence of polysaccharides chosen from the group consisting of hyaluronic acid, keratan, heparin, cellulose, cellulose derivatives, alginic acid, xanthan, carrageenan, chitosan, chondroitin, heparosan, and their biologically acceptable salts, alone or in a mixture.

In one embodiment, the composition according to the invention is characterised in that the macromolecular compound consists of a sequence of hyaluronic acids.

In one embodiment, the composition according to the invention is characterised in that the macromolecular compound consists of a sequence of heparosan.

In one embodiment, the composition according to the invention is characterised in that the macromolecular compound consists of a sequence of polysaccharides chosen from the group consisting of hyaluronic acid, heparosan or their respective salts.

The invention also relates to a process for preparing a composition comprising at least one macromolecular compound consisting of a sequence of polysaccharides, hyaluronic acid for example, and more particularly a preparation process making it possible to obtain a composition comprising at least one macromolecular compound consisting of a sequence of polysaccharides having particular properties such as filterability on a membrane with a porosity of 0.22 μm.

The process for preparing the compositions according to the invention comprises at least the following steps:
a) providing a polysaccharide;
b) providing a crosslinking agent;
c) carrying out one or more crosslinking step(s) in the presence of said polysaccharide and said crosslinking agent;
d) obtaining a crosslinked polysaccharide;
e) implementing one or more step(s) of breaking glycosidic bonds;
f) obtaining a macromolecular compound solution;
g) filtering the macromolecular compound solution through a membrane with a porosity of 0.22 μm.

In one embodiment, the process for preparing the compositions according to the invention comprises at least the following steps:
a) providing a polysaccharide;
b) providing a crosslinking agent;
c) carrying out one or more crosslinking step(s) in the presence of said polysaccharide and said crosslinking agent;
d) obtaining a crosslinked polysaccharide;
e) implementing a step of breaking glycosidic bonds;
f) obtaining a macromolecular compound solution;
g) filtering a sample of the macromolecular compound solution through a membrane with a porosity of 0.22 μm. This is an in-process control (IPC) test.

In one embodiment, the process for preparing the compositions according to the invention comprises at least the following steps:
a) providing a polysaccharide;
b) providing a crosslinking agent;
c) carrying out one or more crosslinking step(s) in the presence of said polysaccharide and said crosslinking agent from a solution of at least one polysaccharide at a concentration greater than or equal to 10% by mass, relative to the total mass of the crosslinking reaction medium, and working at constant temperature or at variable temperature linearly or in stages, said constant or variable temperature being less than or equal to 10° C. for a reaction time of at most 24 hours;
d) obtaining a crosslinked polysaccharide;
e) implementing a step of breaking glycosidic bonds;
f) obtaining a macromolecular compound solution;
g) filtering the macromolecular compound solution through a membrane with a porosity of 0.22 μm.

In one embodiment, the process for preparing the compositions according to the invention comprises at least the following steps:
a) providing a polysaccharide;
b) providing a crosslinking agent;
c) carrying out one or more crosslinking step(s) in the presence of said polysaccharide and said crosslinking agent from a solution of at least one polysaccharide at a concentration greater than or equal to 10% by mass, relative to the total mass of the crosslinking reaction medium, and working at constant temperature or at variable temperature linearly or in stages, said constant or variable temperature being less than or equal to 10° C. for a reaction time of at most 24 hours;
d) obtaining a crosslinked polysaccharide;
e) implementing a step of breaking glycosidic bonds;
f) obtaining a macromolecular compound solution;
g) filtering a sample of the macromolecular compound solution through a membrane with a porosity of 0.22 μm. This is an in-process control (IPC) test,

During step d) we obtain a network made up of different polysaccharides linked together by a divalent radical L, originating from the crosslinking agent.

In one embodiment, the process according to the invention is characterised in that the crosslinking agent is chosen from the group consisting of bis-epoxides, trimetaphosphates, diamines, dialkoxyamines and dihydrazides.

In one embodiment, the process according to the invention is characterised in that the crosslinking agent is 1,4-butanediol diglycidyl ether (BDDE).

In one embodiment, the process according to the invention is characterised in that the crosslinking agent is a trimetaphosphate salt.

In one embodiment, the process according to the invention is characterised in that the crosslinking agent is a diamine.

In one embodiment, the process according to the invention is characterised in that the crosslinking agent is a dihydrazide.

In one embodiment, step e) of breaking glycosidic bonds implemented on at least one crosslinked polysaccharide leads to the majority destruction of the crosslinking network formed during step c).

In one embodiment, step e) of breaking glycosidic bonds implemented on at least one crosslinked polysaccharide leads to the complete destruction of the crosslinking network formed during step c).

During this step e), only glycosidic bonds are broken with the exception of the bonds between the divalent radical L, resulting from the crosslinking agent and the polysaccharide.

In one embodiment, the process according to the invention is characterised in that step e) of breaking glycosidic bonds is carried out by means of a chemical treatment.

In one embodiment, the method according to the invention is characterised in that step e) of breaking glycosidic bonds is carried out by means of a heat treatment.

In one embodiment, the process according to the invention is characterised in that the heat treatment of step e) of breaking glycosidic bonds is carried out by steam autoclaving.

In one embodiment, the method according to the invention is characterised in that step e) of breaking glycosidic bonds is carried out by means of radiation treatment.

In one embodiment, the process according to the invention is characterised in that step e) of breaking glycosidic bonds is carried out by means of an enzymatic treatment.

In one embodiment, the method according to the invention is characterised in that step e) of breaking glycosidic bonds is carried out by means of high-pressure treatment.

The invention also relates to a macromolecular compound of general formula I:

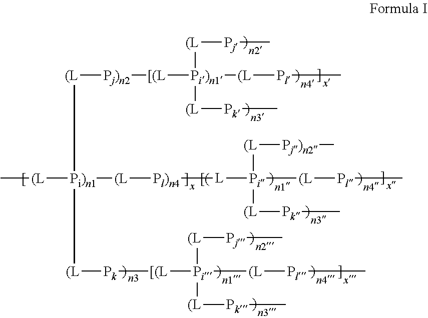

wherein:
Pᵢ, P_{i′}, P_{i″}, P_{i″′}, Pⱼ, P_{j′}, P_{j″}, P_{j″′}, Pₖ, P_{k′}, P_{k″}, P_{k″′}, Pₗ, P_{l′}, P_{l″} and P_{l″′} are identical or different polysaccharides
n1, n1′, n1″, n1″′, n2, n2′, n2″, n2″′, n3, n3′, n3″, n3″′, n4, n4′, n4″ and n4″′ are integers between 0 and 2000 and at least one of n1, n1′, n1″, n1″′, n2, n2′, n2″, n2″′, n3, n3′, n3″, n3″′, n4, n4′, n4″ and n4″′ is greater than or equal to 2
x, x′, x″ and x″′ are integers greater than or equal to 0
L is a divalent radical resulting from a reaction between a crosslinking agent and two reactive functions, each reactive function being carried by two distinct polysaccharide chains
sequences of polysaccharides do not form cyclic structures

In one embodiment, n1, n1′, n1″, n1″′, n2, n2′, n2″, n2″′, n3, n3′, n3″, n3″′, n4, n4′, n4″ and n4″′ are integers between 0 and 1500 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n″′, n2, n2′, n2″, n2″′, n3, n3′, n3″, n3″′, n4, n4′, n4″ and n4″′ are integers between 0 and 1000 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n1″′, n2, n2′, n2″, n2″′, n3, n3′, n3″, n3″′, n4, n4′, n4″ and n4″′ are integers between 0 and 500 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n1″′, n2, n2′, n2″, n2″′, n3, n3′, n3″, n3″′, n4, n4′, n4″ and n4″′ are integers between 0 and 250 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n1″′, n2, n2′, n2″, n2″′, n3, n3′, n3″, n3″′, n4, n4′, n4″ and n4″′ are integers between 0 and 150 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n1″′, n2, n2′, n2″, n2″′, n3, n3′, n3″, n3′″, n4, n4′, n4″ and n4″′ are integers between 0 and 100 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n1″′, n2, n2′, n2″, n2″′, n3, n3′, n3″, n3″′, n4, n4′, n4″ and n4″′ are integers between 0 and 50 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n1″′, n2, n2′, n2″, n2″′, n3, n3′, n3″, n3″′, n4, n4′, n4″ and n4″′ are integers between 0 and 30 and at least one of n1, n1′, n1″, n1″′, n2, n2′, n2″, n2″′, n3, n3′, n3″, n3″′, n4, n4′, n4″ and n4″′ is greater than or equal to 2.

In one embodiment, n1, n1′, n1″, n1″′, n2, n2′, n2″, n2″′, n3′, n3″, n3″′, n4, n4′, n4″, n4″′, x, x′, x″ and x″′ are equal to 0 and n3 is equal to 2. This embodiment represents the macromolecular compound in its simplest form, having the following formula (L-Pₖ)-(L-Pₖ).

In another embodiment, n1, n1′, n1″, n1″′, n2′, n2″, n2″′, n3′, n3″, n3″′, n4, n4′, n4″, n4″′ are equal to 0, n2 and n3 are equal to 1. This embodiment also represents a simple form of the macromolecular compound, having the following formula (L-Pₖ)-(L-Pⱼ).

Consequently, the macromolecular compound according to the invention is a mixed polymer comprising an alternation of polysaccharides linked together by divalent radicals originating from the crosslinker. Indeed, during step e) of breaking glycosidic bonds, the crosslinker becomes a “linker” and it loses its role as a “crosslinker.”

The invention relates to a composition in the form of an aqueous solution, filterable on a membrane with a porosity of 0.22 μm comprising at least one macromolecular compound consisting of sequences of identical or different polysaccharides linked together by a divalent radical L (Linker), characterised in what the macromolecular compound has for general formula I:

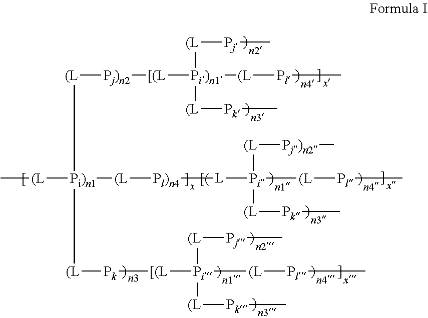

wherein:
Pᵢ, P_{i′}, P_{i″}, P_{i″′}, Pⱼ, P_{j′}, P_{j″}, P_{j″′}, Pₖ, P_{k′}, P_{k″}, P_{k″′}, Pₗ, P_{l′}, P_{l″} and P_{l″′} are identical or different polysaccharides
n1, n1′, n1″, n1″′, n2, n2′, n2″, n2″′, n3, n3′, n3″, n3″′, n4, n4′, n4″ and n4″′ are integers between 0 and 2000 and at least one of n1, n1′, n1″, n1″′, n2, n2′, n2″, n2″′, n3, n3′, n3″, n3″′, n4, n4′, n4″ and n4″′ is greater than or equal to 2
x, x′, x″ and x″′ are integers greater than or equal to 0
L is a divalent radical resulting from a reaction between a crosslinking agent and two reactive functions, each reactive function being carried by two distinct polysaccharide chains
sequences of polysaccharides do not form cyclic structures,

In one embodiment, n1, n1′, n1″, n1″′, n2, n2′, n2″, n2″′, n3, n3′, n3″, n3″′, n4, n4′, n4″ and n4″′ are integers between 0 and 1500 and at least one of n1, n1′, n1″, n1″′, n2, n2′, n2″, n2″′, n3, n3′, n3″, n3″′, n4, n4′, n4″ and n4″′ is greater than or equal to 2.

When the macromolecular compound according to the invention under dry form is hydrated by means of an aqueous solution, an aqueous solution of the macromolecular compound is obtained.

In particular, the macromolecular compound according to the invention does not have swelling capacity.

In the sense of the invention, swelling capacity means the fact that a compound under dry form placed in the presence of an aqueous solution, a saline solution for example, has the capacity to form a gel of remarkable volume, of which more than 80% is retained by filtration on a membrane with a porosity of 0.22 μm. The formation of a gel is characterised by the presence of two phases in the medium: the gel or “soft solid” and the solution or supernatant.

In the presence of water, the macromolecular compound according to the invention does not form a gel.

In particular, the macromolecular compound according to the invention does not form a hydrogel. In the context of the present application, the term “hydrogel” means a gel consisting of a three-dimensional network consisting of at least one compound, capable of absorbing a large quantity of water or aqueous solution and which has particular rheological properties, particularly in terms of viscosity and viscoelasticity.

In one embodiment, said polysaccharide of step a) is chosen from the group of glycosaminoglycans (GAG),

In one embodiment, said polysaccharide from step a) is chosen from the group of chemically modified, oxidized or substituted polysaccharides.

In one embodiment, said polysaccharide is chosen from the group of glycosaminoglycans (GAG), such as chondroitin, keratan, heparin, heparosan or even hyaluronic acid, and mixtures thereof.

In one embodiment, said polysaccharide is chosen from the group consisting of hyaluronic acid, keratan, heparin, cellulose, cellulose derivatives, oxidized cellulose, alginic acid, xanthan, carrageenan, chitosan, chondroitin, heparosan and their biologically acceptable salts, alone or in a mixture.

In one embodiment, said polysaccharide is hyaluronic acid, or one of its biologically acceptable salts, alone or in a mixture.

In the context of the present application, hyaluronic acid, or one of its biologically acceptable salts, alone or in a mixture, are preferred.

In one embodiment, said polysaccharide is chosen from the group consisting of hyaluronic acid, sodium hyaluronate, and mixtures thereof.

In one embodiment, said polysaccharide is hyaluronic acid.

In one embodiment, said polysaccharide is chosen from the group consisting of sodium hyaluronate and potassium hyaluronate.

In one embodiment, said polysaccharide is sodium hyaluronate.

In the context of the present application, sodium hyaluronate is the particularly preferred polysaccharide.

In one embodiment, said polysaccharide is a hyaluronic acid, or one of its salts, chemically modified by substitution.

In one embodiment, said polysaccharide is a hyaluronic acid, or one of its salts, substituted by a group providing lipophilic or hydrating properties, such as substituted hyaluronic acids as described in patent application FR 2 983 483 in the name of the applicant.

In one embodiment, said polysaccharide is heparosan, or one of its biologically acceptable salts, alone or in a mixture.

In the context of the present application, heparosan, or one of its biologically acceptable salts, alone or in a mixture, are preferred.

In one embodiment, said polysaccharide is chosen from the group consisting of heparosan, sodium heparosan, and mixtures thereof.

In one embodiment, said polysaccharide is heparosan.

In one embodiment, said polysaccharide is chosen from the group consisting of sodium heparosan and potassium heparosan.

In one embodiment, said polysaccharide is sodium heparosan.

In the context of the present application, sodium heparosan is the particularly preferred polysaccharide.

In one embodiment, said polysaccharide is a chemically modified heparosan, or one of its salts.

In one embodiment, said polysaccharide is chosen from the group consisting of cellulose and cellulose derivatives.

In one embodiment, said polysaccharide is chosen from the group consisting of cellulose derivatives including in particular hydroxypropylcellulose, hydroxypropylmethylcellulose, ethylmethylcellulose and carboxymethylcellulose.

In one embodiment, said polysaccharide is cellulose.

In one embodiment, said polysaccharide is a cellulose derivative.

In one embodiment, said polysaccharide of step a) of the process for preparing the compositions according to the invention is a mixture of polysaccharides.

In the context of the present application, all the polysaccharides cited can be brought together in the form of a mixture during step a), whether they are polysaccharides of the same nature (for example a mixture of hyaluronic acid having different molecular masses) or of a different nature (for example a mixture of hyaluronic acid and chitosan). During the crosslinking step, there may be co-crosslinking between the different polysaccharides.

In one embodiment, the process for preparing the compositions according to the invention is characterised in that said polysaccharide of step a) is a mixture of hyaluronic acids, or salts of hyaluronic acids.

In one embodiment, the process for preparing the compositions according to the invention is characterised in that said polysaccharide of step a) is a mixture of 2 hyaluronic acids or salts of hyaluronic acids.

In one embodiment, the process for preparing the compositions according to the invention is characterised in that said polysaccharide of step a) is a mixture of 3 hyaluronic acids or salts of hyaluronic acids.

In one embodiment, the process for preparing the compositions according to the invention is characterised in that said polysaccharide of step a) is a mixture of 4 hyaluronic acids or salts of hyaluronic acids.

In one embodiment, the process for preparing the compositions according to the invention is characterised in that said polysaccharide of step a) is a mixture of heparosans, or salts of heparosans.

In one embodiment, the process for preparing the compositions according to the invention is characterised in that said polysaccharide of step a) is a mixture of 2 heparosans or heparosan salts.

In one embodiment, the process for preparing the compositions according to the invention is characterised in that said polysaccharide of step a) is a mixture of 3 heparosans or salts of heparosans.

In one embodiment, the process for preparing the compositions according to the invention is characterised in that said polysaccharide of step a) is a mixture of 4 heparosans or heparosan salts.

In one embodiment, the process for preparing the compositions according to the invention is characterised in that said polysaccharide of step a) is a mixture of hyaluronic acids and heparosans, or their salts.

In one embodiment, the process for preparing the compositions according to the invention is characterised in that said polysaccharide of step a) is a mixture of a hyaluronic acid and a heparosan, or their salts.

In one embodiment, the process for preparing the compositions according to the invention is characterised in that said polysaccharide of step a) is a mixture of 2 hyaluronic acids and a heparosan, or their salts.

In one embodiment, the process for preparing the compositions according to the invention is characterised in that said polysaccharide of step a) is a mixture of a hyaluronic acid and 2 heparosans, or their salts.

In one embodiment, the process for preparing the compositions according to the invention is characterised in that said polysaccharide of step a) is a mixture of 2 hyaluronic acids and 2 heparosans, or their salts.

In one embodiment, the process for preparing the compositions according to the invention is characterised in that said polysaccharide of step a) is a mixture of hyaluronic acids or their salts and celluloses.

In one embodiment, the process for preparing the compositions according to the invention is characterised in that said polysaccharide of step a) is a mixture of a hyaluronic acid or its salt and a cellulose.

In one embodiment, the process for preparing the compositions according to the invention is characterised in that said polysaccharide of step a) is a mixture of 2 hyaluronic acids or their salts and a cellulose.

In one embodiment, the process for preparing the compositions according to the invention is characterised in that said polysaccharide of step a) is a mixture of a hyaluronic acid or its salt and 2 celluloses.

In one embodiment, the process for preparing the compositions according to the invention is characterised in that said polysaccharide of step a) is a mixture of 2 hyaluronic acids or their salts and 2 celluloses.

In the context of the present application, Mw or “molecular mass” is called the weight average molecular mass of the polysaccharides, measured in Daltons.

In one embodiment, said polysaccharide or one of its salts has a molecular mass of 0.01 MDa to 10 MDa (0.01 MDa≤Mw≤10 MDa),

In one embodiment, said polysaccharide or one of its salts has a molecular mass of between 0.01 MDa and 8 MDa.

In one embodiment, said polysaccharide or one of its salts has a molecular mass of between 0.01 MDa and 5 MDa.

In one embodiment, said polysaccharide or one of its salts has a molecular mass of between 0.01 MDa and 3.5 MDa.

In one embodiment, said polysaccharide or one of its salts has a molecular mass of between 0.5 MDa and 3.5 MDa.

In one embodiment, said polysaccharide or one of its salts has a molecular mass of between 2.75 MDa and 3.25 MDa.

In one embodiment, said polysaccharide or one of its salts has a molecular mass of between 0.75 MDa and 1.25 MDa.

In one embodiment, said polysaccharide or one of its salts has a molecular mass of between 2 MDa and 5 MDa.

In one embodiment, said polysaccharide or one of its salts has a molecular mass of between 2 MDa and 4 MDa.

In one embodiment, said polysaccharide or one of its salts has a molecular mass of between 0.5 MDa and 2 MDa.

In one embodiment, said polysaccharide acid or one of its salts has a molecular mass of between 0.5 MDa and 1.5 MDa.

In one embodiment, said hyaluronic acid or one of its salts has a molecular mass of between 0.01 MDa and 10 MDa.

In one embodiment, said hyaluronic acid or one of its salts has a molecular mass of between 0.01 MDa and 5 MDa.

In one embodiment, said hyaluronic acid or one of its salts has a molecular mass of between 0.01 MDa and 3.5 MDa.

In one embodiment, said hyaluronic acid or one of its salts has a molecular mass of between 0.5 MDa and 3.5 MDa.

In one embodiment, said hyaluronic acid or one of its salts has a molecular mass of between 2.75 MDa and 3.25 MDa.

In one embodiment, said hyaluronic acid or one of its salts has a molecular mass of between 0.75 MDa and 1.25 MDa.

In one embodiment, said hyaluronic acid or one of its salts has a molecular mass of between 2 MDa and 5 MDa.

In one embodiment, said hyaluronic acid or one of its salts has a molecular mass of between 2 MDa and 4 MDa.

In one embodiment, said hyaluronic acid or one of its salts has a molecular mass of between 0,5 MDa and 2 MDa.

In one embodiment, said hyaluronic acid or one of its salts has a molecular mass of between 0.5 MDa and 1.5 MDa.

In one embodiment, said heparosan or one of its salts has a molecular mass of between 0.01 MDa and 10 MDa.

In one embodiment, said heparosan or one of its salts has a molecular mass of between 0.01 MDa and 5 MDa.

In one embodiment, said heparosan or one of its salts has a molecular mass of between 0.02 MDa and 3 MDa.

In one embodiment, said heparosan or one of its salts has a molecular mass of between 0.02 MDa and 2 MDa.

For the preparation of the compositions according to the invention, one or more crosslinking steps c) are implemented between a polysaccharide and at least one crosslinking agent.

In one embodiment, the process for preparing the compositions according to the invention is characterised in that the bringing together of said polysaccharide and at least one crosslinking agent takes place in a solvent.

In one embodiment, the process for preparing the compositions according to the invention is characterised in that said at least one crosslinking agent of step c) is chosen from the group consisting of a bis-epoxy such as ether ethyleneglycoldiglycidyl, 1,4-butanediol diglycidyl ether (BDDE), 1,2,3,4-diepoxybutane or 1,2,7,8-diepoxyoctane, a dialkylsulfone, divinylsulfone, formaldehyde, epichlorohydrin or even glutaraldehyde, carbodiimides such as 1-ethyl-3-[3-dimethylaminopropyl]carbodiimide hydrochloride (EDC), trimetaphosphates, such as sodium trimetaphosphate, calcium trimetaphosphate, or even barium trimetaphosphate.

In one embodiment, the process for preparing the compositions according to the invention is characterised in that said at least one crosslinking agent of step c) is chosen from the group consisting of a bis-epoxy such as ether ethylene glycol diglycidyl, 1,4-butanediol diglycidyl ether (BDDE), 1,2,3,4-diepoxybutane or 1,2,7,8-diepoxyoctane, trimetaphosphates, such as sodium trimetaphosphate, calcium trimetaphosphate, or even barium trimetaphosphate.

In one embodiment, the process for preparing the compositions according to the invention is characterised in that said at least one crosslinking agent of step c) is chosen from the group consisting of ethyleneglycoldiglycidyl ether, 1,4-butanediol diglycidyl ether (BDDE), 1,2,3,4-diepoxybutane or 1,2,7,8-diepoxyoctane.

In one embodiment, the process for preparing the compositions according to the invention is characterised in that said at least one crosslinking agent of step c) is chosen from the group consisting of difunctional polyethylene glycol (PEG) carrying an epoxide at each end of the polymer chain.

In one embodiment, the process for preparing the compositions according to the invention is characterised in that said at least one crosslinking agent of step c) is chosen from the group consisting of difunctional polyethylene glycol (PEG) bearing an epoxy group at each end of the polymer chain chosen from the group consisting of polyethylene glycol diglycidyl ether, polypropylene glycol diglycidyl ether and polytetramethylene glycol diglycidyl ether.

In one embodiment, the process for preparing the compositions according to the invention is characterised in that said at least one crosslinking agent of step c) is chosen from the group consisting of trimetaphosphates, such as sodium trimetaphosphate, calcium trimetaphosphate, or even barium trimetaphosphate.

In one embodiment, the process for preparing the compositions according to the invention is characterised in that said at least one crosslinking agent from step c) is chosen from the group consisting of epoxides, for example 1,4-butanediol diglycidyl ether (BDDE), epihalohydrins, divinylsulfone (DVS).

In one embodiment, the process for preparing the compositions according to the invention is characterised in that said at least one crosslinking agent from step c) is divinylsulfone (DVS).

In one embodiment, the process for preparing the compositions according to the invention is characterised in that said at least one crosslinking agent from step c) is 1,4-butanediol diglycidyl ether (BDDE).

In the context of this application, BDDE is particularly preferred.

In one embodiment, the process for preparing the compositions according to the invention is characterised in that said at least one crosslinking agent of step c) is chosen from compounds carrying two functions chosen from the group consisting of amine functions, alkoxyamines and hydrazides.

In one embodiment, the process for preparing the compositions according to the invention is characterised in that said at least one crosslinking agent from step c) is chosen from diamines.

In one embodiment, the diamine used as a crosslinking agent in step c) of the process for preparing the compositions according to the invention is chosen from the group consisting of diaminotrehalose, diaminosucrose, chitobiose, diaminolactose and diaminoraffinose.

In one embodiment, the diamine used as crosslinking agent in step c) of the process for preparing the compositions according to the invention is diaminotrehalose.

In one embodiment, the process for preparing the compositions according to the invention is characterised in that said at least one crosslinking agent from step c) is chosen from dialkoxyamines.

In one embodiment, the process for preparing the compositions according to the invention is characterised in that said at least one crosslinking agent from step c) is chosen from dihydrazides.

In one embodiment, the dihydrazide used as crosslinking agent in step c) of the process for preparing the compositions according to the invention is adipic acid dihydrazide.

In one embodiment, the crosslinking step or each of the crosslinking steps c) requires that said polysaccharide be dissolved beforehand.

In one embodiment, the polysaccharide is added in solid form to a solution to dissolve said polysaccharide.

In one embodiment, a solution is added to the polysaccharide in solid form to dissolve said polysaccharide.

In one embodiment, the crosslinking step or each of the steps c) does not require said polysaccharide to be dissolved.

The polysaccharide is dissolved by adding water or an aqueous saline solution, for example a phosphate buffer solution, for example PBS, or by adding a sodium hydroxide or acid solution to obtain the pH compatible with the implementation of the crosslinking step or each of the steps.

In one embodiment, the polysaccharide is dissolved by adding water or an aqueous saline solution, for example a phosphate buffer solution, for example PBS, further comprising at least one active ingredient, for example an antioxidant and/or a local anaesthetic, or by adding a solution of sodium hydroxide or acid to obtain the pH compatible with the implementation of the step or each of the crosslinking steps.

In one embodiment, the process for obtaining the compositions according to the invention is characterised in that, at the latest during step c), a step of adjusting the pH to a crosslinking pH is carried out.

The pH adjustment is carried out by adding a mineral acid solution preferably, for example hydrochloric acid or a mineral base preferably, for example soda or potash, said acids and bases being added in a quantity making it possible to achieve the targeted crosslinking pH.

In one embodiment, during step or each of the crosslinking steps c), a step of adjusting the pH to a crosslinking pH adapted to said crosslinking agent is carried out.

In one embodiment, at the latest during the crosslinking step or each of the crosslinking steps c), a pH adjustment step is carried out to a crosslinking pH greater than 10.

In one embodiment, at the latest during the crosslinking step or each of the crosslinking steps c), a pH adjustment step is carried out to a crosslinking pH lower than 3.

In one embodiment, the crosslinking pH targeted during the crosslinking step or each of the crosslinking steps c) is obtained by adding a sodium hydroxide solution with a concentration of at most 0.25 N or 1% by mass.

In one embodiment, at the latest during the crosslinking step or each of the crosslinking steps c), a pH adjustment step is carried out at a crosslinking pH greater than 10, said crosslinking agent being BDDE.

In one embodiment, at the latest during the crosslinking step or each of the crosslinking steps c), a pH adjustment step is carried out at a crosslinking pH lower than 3, said crosslinking agent being BDDE.

In one embodiment, at the latest during the crosslinking step or each of the crosslinking steps c), a step of adjusting the pH to a crosslinking pH is carried out, said crosslinking pH being greater than 10.

Crosslinking begins when the following 3 conditions are met: presence of the polysaccharide, presence of the crosslinking agent, reaction medium at an appropriate pH.

In one embodiment, the crosslinking step or each of the crosslinking steps c) is characterised in that the initiation of the crosslinking is caused by the addition of said crosslinking agent.

In one embodiment, the crosslinking step or each of the crosslinking steps c) is characterised in that the initiation of the crosslinking is caused by the addition of said polysaccharide.

In one embodiment, the crosslinking step or each of the crosslinking steps c) is characterised in that the initiation of the crosslinking is caused by the application of a crosslinking pH.

In one embodiment, the process for preparing the compositions according to the invention is characterised in that after step c), a step of adjusting the pH to a pH of between 6 and 8 is carried out.

Depending on the pH of the reaction medium at the end of the crosslinking reaction of step c), the pH adjustment is carried out by adding a preferably mineral acid solution, for example hydrochloric acid or a mineral base preferably, for example soda or potash, said acids and bases being added in a quantity making it possible to reach a pH of between 6 and 8.

In one embodiment, the process for preparing the compositions according to the invention is characterised in that after step c), a step of adjusting the pH to a pH of between 6 and 8 is carried out by adding at least one acid being hydrochloric acid (HCl).

The process for preparing the compositions according to the invention is characterised in that the step or each of the crosslinking steps c) is carried out at constant temperature or at variable temperature linearly or in stages, said constant or variable temperature being less than or equal to 50° C.

The process for preparing the compositions according to the invention is characterised in that the step or each of the crosslinking steps c) is carried out at constant temperature or at variable temperature linearly or in stages, said constant or variable temperature being less than or equal to 30° C.

The process for preparing the compositions according to the invention is characterised in that the step or each of the crosslinking steps c) is carried out at constant temperature or at variable temperature linearly or in stages, said constant or variable temperature being less than or equal to 20° C.

The process for preparing the compositions according to the invention is characterised in that the step or each of the crosslinking steps c) is carried out at constant temperature or at variable temperature linearly or in stages, said constant or variable temperature being less than or equal to 15° C.

The process for preparing the compositions according to the invention is characterised in that the step or each of the crosslinking steps c) is carried out at constant temperature or at variable temperature linearly or in stages, said constant or variable temperature being less than or equal to 10° C.

The process for preparing the compositions according to the invention is characterised in that the step or each of the crosslinking steps c) is carried out at constant temperature or at variable temperature linearly or in stages, said constant or variable temperature being less than or equal to 5° C.

By solidification temperature of the reaction medium, we mean the temperature at which the medium becomes solid. For an aqueous medium, this temperature will be at a temperature of 0° C., or slightly lower depending on the salt concentration of said medium.

In one embodiment, the step or each of the crosslinking steps c) is carried out at constant temperature or at variable temperature linearly or in stages, said constant or variable temperature being between the solidification temperature and 10° C.

In one embodiment, the step or each of the crosslinking steps c) is carried out at constant temperature or at variable temperature linearly or in stages, said constant or variable temperature being between the solidification temperature and 5° C.

In one embodiment, the step or each of the crosslinking steps c) is carried out at constant temperature at 50° C.

In one embodiment, the step or each of the crosslinking steps c) is carried out at constant temperature at 30° C.

In one embodiment, the step or each of the crosslinking steps c) is carried out at constant temperature at 20° C.

In one embodiment, the step or each of the crosslinking steps c) is carried out at constant temperature at 15° C.

In one embodiment, step or each of the crosslinking steps c) is carried out at constant temperature at 9° C.

In one embodiment, the step or each of the crosslinking steps c) is carried out at constant temperature at 5° C.

In one embodiment, the step or each of the crosslinking steps c) is carried out at a constant temperature of 2° C.

In one embodiment, before the crosslinking step or each of the crosslinking steps c), a cooling step is carried out at the crosslinking temperature.

In one embodiment, the process for preparing the compositions according to the invention is characterised in that said at least one crosslinking agent of step c) is 1,4-butanediol diglycidyl ether (BDDE), and said step c)) is carried out at a pH greater than 10 and at a temperature less than or equal to 50° C.

In one embodiment, the process for preparing the compositions according to the invention is characterised in that said at least one crosslinking agent of step c) is 1,4-butanediol diglycidyl ether (BDDE), and said step c)) is carried out at a pH greater than 10 and at a temperature less than or equal to 30° C.

In one embodiment, the process for preparing the compositions according to the invention is characterised in that said at least one crosslinking agent of step c) is 1,4-butanediol diglycidyl ether (BDDE), and said step c)) is carried out at a pH greater than 10 and at a temperature less than or equal to 20° C.

In one embodiment, the process for preparing the compositions according to the invention is characterised in that said at least one crosslinking agent of step c) is 1,4-butanediol diglycidyl ether (BDDE), and said step c)) is carried out at a pH greater than 10 and at a temperature less than or equal to 15° C.

In one embodiment, the process for preparing the compositions according to the invention is characterised in that said at least one crosslinking agent of step c) is 1,4-butanediol diglycidyl ether (BDDE), and said step c)) is carried out at a pH greater than 10 and at a temperature less than or equal to 10° C.

In one embodiment, the process for preparing the compositions according to the invention is characterised in that said at least one crosslinking agent of step c) is 1,4-butanediol diglycidyl ether (BDDE), and said step c)) is carried out at a pH greater than 10 and at a temperature less than or equal to 5° C.

In one embodiment, the process for preparing the compositions according to the invention is characterised in that said at least one crosslinking agent of step c) is 1,4-butanediol diglycidyl ether (BDDE), and said step c)) is carried out at a pH greater than 10 and at a temperature less than or equal to 2° C.

In one embodiment, the process for preparing the compositions according to the invention is characterised in that crosslinking step c) has a duration of between 10 min and 26 hours.

In one embodiment, the process for preparing the compositions according to the invention is characterised in that crosslinking step c) has a duration of between 10 min and 18 hours.

In one embodiment, the process for preparing the compositions according to the invention is characterised in that crosslinking step c) has a duration of between 10 min and 12 hours.

In one embodiment, the process for preparing the compositions according to the invention is characterised in that crosslinking step c) has a duration of between 10 min and 5 hours.

In one embodiment, the process for preparing the compositions according to the invention is characterised in that crosslinking step c) has a duration of between 10 min and 3 hours.

In one embodiment, the process for preparing the compositions according to the invention is characterised in that crosslinking step c) has a duration of between 30 min and 3 hours.

In one embodiment, the process for preparing the compositions according to the invention is characterised in that the crosslinking step c) is carried out at 2° C. for a period of 24 hours.

In one embodiment, the process for preparing the compositions according to the invention is characterised in that said at least one crosslinking agent of step c) is 1,4-butanediol diglycidyl ether (BDDE), and said step c) is carried out at a pH greater than 10, at a temperature of 2° C. for a period of 24 hours.

In one embodiment, the process for preparing the compositions according to the invention is characterised in that the crosslinking step c) is carried out at 9° C. for a period of 3 hours.

In one embodiment, the process for preparing the compositions according to the invention is characterised in that said at least one crosslinking agent of step c) is 1,4-butanediol diglycidyl ether (BDDE), and said step c) is carried out at a pH greater than 10, at a temperature of 9°° C. for a period of 3 hours.

When several successive crosslinking reactions are carried out in step c) of the process for preparing the compositions according to the invention, the times mentioned are the total times (sum of the times of the successive crosslinking reactions).

In one embodiment, during step c), the implementation of the crosslinking step(s) in the presence of said polysaccharide and said crosslinking agent takes place in a medium in which said polysaccharide is hydrated and/or swollen by the addition of water or an aqueous saline solution, for example a phosphate buffer solution, for example PBS.

In one embodiment, during step c), the implementation of the crosslinking step(s) in the presence of said polysaccharide and said crosslinking agent takes place in a medium in which said polysaccharide is hydrated and/or swollen by the addition of water or an aqueous saline solution, for example a phosphate buffer solution, for example PBS, further comprising at least one active ingredient, such as an antioxidant and/or a local anaesthetic.

During step c) of the process for preparing the compositions according to the invention, the crosslinking rate (X) can be calculated theoretically using the following formula:

$$X = \frac{\begin{matrix}number of moles of crosslinking agent introduced \\ into the reaction medium\end{matrix}}{\begin{matrix}number of moles of repeating units ( disaccharide pattern ) \\ introduced into the reaction medium\end{matrix}}$$

Thus, for example if a medium comprises 100 disaccharide units, and said medium also comprises 10 molecules of crosslinking agent, then the crosslinking rate (X) will be as follows: X=10/100=0.1. This rate of crosslinking is therefore influenced neither by the degree of polymerization, nor by the molecular mass of the polysaccharide chosen, nor by the proportion of crosslinking agent which actually reacts with at least one function of the polysaccharide. This is a theoretical determination taking into account only the quantities of crosslinking agent and repeating units brought together.

In one embodiment, the crosslinking rate X is between 0.001 and 0.20.

In one embodiment, the crosslinking rate X is between 0.01 and 0.15.

In one embodiment, the crosslinking rate X is between 0.01 and 0.12.

In one embodiment, the crosslinking rate X is between 0.03 and 0.10.

In one embodiment, the crosslinking rate X is between 0.04 and 0.08.

Crosslinking can also be assessed, a posteriori (after crosslinking), by means of the degree of modification (Mod). The Mod therefore takes into account, contrary to the crosslinking rate X, the proportion of crosslinking agent which actually reacts with at least one function of the polysaccharide.

The degree of modification can be expressed as follows:

$$Mod ( \% ) = \frac{\begin{matrix}number of moles of crosslinking agent linked by at least one \\ covalent bond to at least one disaccharide unit\end{matrix}}{\begin{matrix}number of moles of repeating units \\ present in the reaction medium\end{matrix}} * 100$$

The repeating unit (or monomer) is, when the polysaccharide is hyaluronic acid, a disaccharide unit.

The determination of the values in the numerator and the denominator depends on the polysaccharide chosen and the crosslinking agent chosen, and are well known to those skilled in the art. For example, in the particular case of a formulation based on hyaluronic acid crosslinked with BDDE, the method described in the publication L. Nord, A. Emilson, C. Sturesson, A H Kenne, Degree of Modification of Hyaluronic Acid Dermal Fillers, 18th Congress of the EADV, Berlin, 2009 can be used.

In the particular case of a formulation based on hyaluronic acid crosslinked with BDDE, the degree of modification can be expressed as follows:

$$Mod ( \% ) = \frac{\begin{matrix}number of moles of BDDE linked by at least one covalent \\ bond to at least one disaccharide unit of hyaluronic acid\end{matrix}}{\begin{matrix}number of moles of repeating \\ units ( disaccharide unit of hyaluronic acid ) \\ present in the reaction medium\end{matrix}} * 100$$

For example, a formulation based on hyaluronic acid crosslinked with BDDE having a Mod of 1% means that it has one BDDE molecule (mono-linked or doubly linked) for every 100 disaccharide units.

In one embodiment, the degree of modification of said crosslinked polysaccharide is less than 5%.

In one embodiment, the degree of modification of said crosslinked polysaccharide is less than 4%.

In one embodiment, the degree of modification of said crosslinked polysaccharide is less than 3%.

In one embodiment, the degree of modification of said crosslinked polysaccharide is less than 2%.

In one embodiment, during step c), implementation of the crosslinking step(s) in the presence of said polysaccharide and said crosslinking agent, the polysaccharide concentration is at least 1% by mass, relative to the total mass of the crosslinking reaction medium.

In one embodiment, during step c), implementation of the crosslinking step(s) in the presence of said polysaccharide and said crosslinking agent, the polysaccharide concentration is at least 5% by mass, relative to the total mass of the crosslinking reaction medium.

In one embodiment, during step c), implementation of the crosslinking step(s) in the presence of said polysaccharide and said crosslinking agent, the polysaccharide concentration is at least 10% by mass, relative to the total mass of the crosslinking reaction medium.

In one embodiment, during step c), implementation of the crosslinking step(s) in the presence of said polysaccharide and said crosslinking agent, the polysaccharide concentration is at least 15% by mass, relative to the total mass of the crosslinking reaction medium.

In one embodiment, during step c), implementation of the crosslinking step(s) in the presence of said polysaccharide and said crosslinking agent, the polysaccharide concentration is between 1 and 60% by mass, relative to the total mass of the crosslinking reaction medium.

In one embodiment, during step c), implementation of the crosslinking step(s) in the presence of said polysaccharide and said crosslinking agent, the polysaccharide concentration is between 5 and 60% by mass, relative to the total mass of the crosslinking reaction medium.

In one embodiment, during step c), implementation of the crosslinking step(s) in the presence of said polysaccharide and said crosslinking agent, the polysaccharide concentration is between 10 and 60% by mass, relative to the total mass of the crosslinking reaction medium.

In one embodiment, during step c), implementation of the crosslinking step(s) in the presence of said polysaccharide and said crosslinking agent, the polysaccharide concentration is between 10 and 40% by mass, relative to the total mass of the reaction medium.

In one embodiment, during step c), implementation of the crosslinking step(s) in the presence of said polysaccharide and said crosslinking agent, the polysaccharide concentration is between 10 and 25% by mass, relative to the total mass of the reaction medium.

In one embodiment, during step c), implementation of the crosslinking step(s) in the presence of said polysaccharide and said crosslinking agent, the polysaccharide concentration is between 15 and 25% by mass, relative to the total mass of the reaction medium.

In one embodiment, during step c), implementation of the crosslinking step(s) in the presence of hyaluronic acid, or one of its biologically acceptable salts, alone or in a mixture, and of said crosslinking agent in a crosslinking reaction medium, the concentration of hyaluronic acid is between 1 and 60% by mass, relative to the total mass of the reaction medium.

In one embodiment, during step c), implementation of the crosslinking step(s) in the presence of hyaluronic acid, or one of its biologically acceptable salts, alone or in a mixture, and of said crosslinking agent in a crosslinking reaction medium, the concentration of hyaluronic acid is between 5 and 60% by weight, relative to the total mass of the reaction medium.

In one embodiment, during step c), implementation of the crosslinking step(s) in the presence of hyaluronic acid, or one of its biologically acceptable salts, alone or in a mixture, and of said crosslinking agent in a crosslinking reaction medium, the concentration of hyaluronic acid is between 10 and 60% by weight, relative to the total mass of the reaction medium.

In one embodiment, during step c), implementation of the crosslinking step(s) in the presence of hyaluronic acid, or one of its biologically acceptable salts, alone or in a mixture, and of said crosslinking agent, the concentration of hyaluronic acid is between 10 and 40% by mass, relative to the total mass of the reaction medium.

In one embodiment, during step c), implementation of the crosslinking step(s) in the presence of hyaluronic acid, or one of its biologically acceptable salts, alone or in a mixture, and of said crosslinking agent, the concentration of hyaluronic acid is between 10 and 25% by mass, relative to the total mass of the reaction medium.

In one embodiment, during step c), implementation of the crosslinking step(s) in the presence of hyaluronic acid, or one of its biologically acceptable salts, alone or in a mixture, and of said crosslinking agent, the concentration of hyaluronic acid is between 15 and 25% by mass, relative to the total mass of the reaction medium.

In one embodiment, during step c), implementation of the crosslinking step(s) in the presence of heparosan, or one of its biologically acceptable salts, alone or in a mixture, and said crosslinking agent in a crosslinking reaction medium, the heparosan concentration is between 1 and 60% by weight, relative to the total mass of the reaction medium.

In one embodiment, during step c), implementation of the crosslinking step(s) in the presence of heparosan, or one of its biologically acceptable salts, alone or in a mixture, and said crosslinking agent in a crosslinking reaction medium, the heparosan concentration is between 5 and 60% by weight, relative to the total mass of the reaction medium.

In one embodiment, during step c), implementation of the crosslinking step(s) in the presence of heparosan, or one of its biologically acceptable salts, alone or in a mixture, and said crosslinking agent in a crosslinking reaction medium, the heparosan concentration is between 10 and 60% by weight, relative to the total mass of the reaction medium.

In one embodiment, during step c), implementation of the crosslinking step(s) in the presence of heparosan, or one of its biologically acceptable salts, alone or in a mixture, and said crosslinking agent the concentration of heparosan is between 10 and 40% by mass, relative to the total mass of the reaction medium.

In one embodiment, during step c), implementation of the crosslinking step(s) in the presence of heparosan, or one of its biologically acceptable salts, alone or in a mixture, and said crosslinking agent the concentration of heparosan is between 10 and 25% by weight, relative to the total mass of the reaction medium.

In one embodiment, during step c), implementation of the crosslinking step(s) in the presence of heparosan, or one of its biologically acceptable salts, alone or in a mixture, and said crosslinking agent the concentration of heparosan is between 15 and 25% by weight, relative to the total mass of the reaction medium.

In one embodiment, during step c), implementation of the crosslinking step(s) in the presence of said polysaccharide and said crosslinking agent, the crosslinking reaction medium comprises sodium hydroxide (NaOH).

In one embodiment, during step c), implementation of the crosslinking step(s) in the presence of said polysaccharide and said crosslinking agent, the soda concentration is between 0.5 and 1.5% by mass, relative to the total mass of the reaction medium.

In one embodiment, during step c), implementation of the crosslinking step(s) in the presence of said polysaccharide and said crosslinking agent, the soda concentration is between 0.5 and 1% in mass, relative to the total mass of the reaction medium,

In one embodiment, during step c), implementation of the crosslinking step(s) in the presence of said polysaccharide and said crosslinking agent, the soda concentration is between 0.7 and 0.9% in mass, relative to the total mass of the reaction medium.

In one embodiment, during step c), implementation of the crosslinking step(s) in the presence of said polysaccharide and said crosslinking agent, the crosslinking reaction medium comprises hydrochloric acid (HCl).

In one embodiment, during step c), implementation of the crosslinking step(s) in the presence of said polysaccharide and said crosslinking agent, the concentration of hydrochloric acid is between 0.001 and 0.05 M in the reaction medium.

In one embodiment, crosslinking step c) comprises at least one activation step of chemical functions carried by the polysaccharide.

In one embodiment, crosslinking step c) comprises at least one step of activating carboxylic acid functions carried by the polysaccharide.

In one embodiment, the step of activating the carboxylic acid functions carried by the polysaccharide is carried out using a coupling agent used in peptide chemistry.

In one embodiment, the step of activating the carboxylic acid functions carried by the polysaccharide is carried out using a coupling agent used in peptide chemistry chosen from the group of triazine, carbodiimide, imidazolium type coupling agents, as well as Oxyma and COMU.

In one embodiment, the triazine type coupling agent is chosen from the group consisting of 4-(4,6-dimethoxy[1.3.5]triazin-2-yl)-4-methylmorpholinium chloride (DMTMM) and 2-Chloro-4,6-dimethoxy-1,3,5-triazine (CMT).

In one embodiment, the coupling agent is 4-(4,6-dimethoxy[1.3.5]triazin-2-yl)-4-methylmorpholinium chloride (DMTMM).

In one embodiment, the process for preparing the compositions according to the invention is characterised in that before step d), a step of eliminating said crosslinking agent is carried out.

In one embodiment, the process for preparing the compositions according to the invention is characterised in that before step d), a step of purification of the crosslinked polysaccharide is carried out.

In one embodiment, the process for preparing the compositions according to the invention is characterised in that before step d), a step of purification of the crosslinked polysaccharide is carried out by dialysis.

In one embodiment, the process for preparing the compositions according to the invention is characterised in that before step d), a step of purifying the crosslinked polysaccharide is carried out by dialysis using a solution or a dialysis solvent chosen from the group consisting of phosphate buffers, for example PBS, and water.

In one embodiment, the process for preparing the compositions according to the invention is characterised in that before step d), a step of purifying the crosslinked polysaccharide is carried out by dialysis using a solution or a dialysis solvent chosen from the group consisting of phosphate buffers, for example PBS, further comprising at least one active ingredient, for example an antioxidant and/or a local anaesthetic, and water.

In one embodiment, the process for preparing the compositions according to the invention is characterised in that before step d), a step of purification of the crosslinked polysaccharide is carried out by washing,

In one embodiment, a step of purifying the crosslinked polysaccharide is carried out by washing using a solution or a solvent chosen from the group consisting of phosphate buffers, for example PBS, and water.

In one embodiment, a step of purifying the crosslinked polysaccharide is carried out by washing using a solution or a solvent chosen from the group consisting of phosphate buffers, for example PBS, further comprising at least one active ingredient, for example an antioxidant and/or local anaesthetic, and water.

In one embodiment, the process for preparing the compositions according to the invention is characterised in that before step d), a step of purification of the crosslinked polysaccharide is carried out by precipitation in salified form.

In one embodiment, the process for preparing the compositions according to the invention is characterised in that before step d), a step of purification of the crosslinked polysaccharide is carried out by precipitation from a solution obtained after addition of PBS or NaCl salt.

In one embodiment, the process for preparing the compositions according to the invention is characterised in that before step d), a step of purification of the crosslinked polysaccharide is carried out by precipitation from a solution obtained after addition of PBS or NaCl salt, further comprising at least one active ingredient, for example an antioxidant and/or a local anaesthetic.

In one embodiment, the step of purifying the crosslinked polysaccharide by precipitation is carried out by adding at least one organic solvent to the crosslinked polysaccharide.

In one embodiment, the at least one organic solvent added to the crosslinked polysaccharide in the purification step by precipitation is chosen from the group of alcohols.

In one embodiment, the alcohol added to the crosslinked polysaccharide in the purification step by precipitation is ethanol.

In one embodiment, the step of purifying the crosslinked polysaccharide by precipitation takes place in a hydroalcoholic mixture.

In one embodiment, the precipitated crosslinked polysaccharide is isolated by filtration.

In one embodiment, the crosslinked polysaccharide precipitated and isolated by filtration is washed.

In one embodiment, the crosslinked polysaccharide precipitated and isolated by filtration is dried.

In one embodiment, the precipitated crosslinked polysaccharide and isolated by filtration is dried under atmosphere and room temperature.

In one embodiment, the precipitated crosslinked polysaccharide and recovered by filtration is dried under a vacuum at room temperature.

In one embodiment, the crosslinked polysaccharide is lyophilized.

In one embodiment, the process for preparing the compositions according to the invention is characterised in that before step d), a step of dilution of the crosslinked polysaccharide is carried out by means of a solution or a solvent chosen in the group consisting of phosphate buffers such as PBS and water.

In one embodiment, the process for preparing the compositions according to the invention is characterised in that before step d), a step of dilution of the crosslinked polysaccharide is carried out by means of a solution or a solvent chosen in the group consisting of phosphate buffers, for example PBS, and water, further comprising at least one active ingredient, for example an antioxidant and/or a local anaesthetic, and water.

In one embodiment, the process for preparing the compositions according to the invention is characterised in that before step d), a step of dilution of the crosslinked polysaccharide is carried out to obtain a concentration of crosslinked polysaccharide of between 2 mg/g and 200 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of between 2 mg/g and 75 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of between 5 mg/g and 50 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of between 10 mg/g and 40 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of approximately 100 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of approximately 80 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of approximately 60 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of approximately 40 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of approximately 20 mg/g, relative to the total mass of said compositions.

In one embodiment, the process for preparing the compositions according to the invention is characterised in that before step d), a step of dilution of the crosslinked polysaccharide is carried out to obtain a concentration of crosslinked polysaccharide of approximately 10 mg/g, relative to the total mass of said compositions.

In one embodiment, the polysaccharide obtained in step d) has a tangent Tan Δ (Tn δ)≥0.50 before the bond-breaking step of step e).

In one embodiment, the polysaccharide obtained in step d) has a tangent Tan Δ (Tn δ)≥0.70 before the bond-breaking step of step e).

The process for preparing the compositions according to the invention comprises at least one step of breaking glycosidic bonds, step e), implemented on at least one crosslinked polysaccharide and making it possible to obtain the macromolecular compound according to the invention.

In one embodiment, step e) of breaking glycosidic bonds implemented on at least one crosslinked polysaccharide is based on a controlled degradation mechanism.

In one embodiment, step e) of breaking glycosidic bonds implemented on at least one crosslinked polysaccharide is carried out by means of a treatment chosen from the group of chemical treatments, treatments by enzymes, treatments by radiation and heat treatments.

In one embodiment, step e) of breaking glycosidic bonds carried out on at least one crosslinked polysaccharide is carried out by means of a chemical treatment.

In one embodiment, this chemical treatment consists of exposing the at least one crosslinked polysaccharide to an aqueous solution at basic pH.

In one embodiment, the aqueous solution at basic pH is an aqueous solution of sodium hydroxide (soda).

In one embodiment, this chemical treatment consists of exposing the at least one crosslinked polysaccharide to an aqueous solution with an acidic pH.

In one embodiment, the aqueous solution at acidic pH contains an acid chosen from the group consisting of acetic acid, hydrochloric acid, sulfuric acid and phosphoric acid.

In one embodiment, the aqueous solution with acidic pH is an aqueous solution of acetic acid.

In one embodiment, the aqueous solution with acidic pH is an aqueous solution of hydrochloric acid.

In one embodiment, the aqueous solution with acidic pH is an aqueous solution of sulfuric acid.

In one embodiment, the aqueous solution with acidic pH is an aqueous solution of phosphoric acid.

In one embodiment, this chemical treatment consists of exposing the at least one crosslinked polysaccharide to an oxidising agent.

In one embodiment, the oxidising agent is chosen from the group consisting of hydrogen peroxide and sodium hypochlorite.

In one embodiment, the oxidising agent is hydrogen peroxide.

In one embodiment, the oxidising agent is sodium hypochlorite.

In one embodiment, this chemical treatment consists of subjecting the crosslinked polysaccharide to an oxidative reductive depolymerisation process.

In one embodiment, the oxidative reductive depolymerisation process is carried out in the presence of at least one of the compounds Fe₂⁺, Fe₃⁺, ascorbic acid and H₂O₂

In one embodiment, this chemical treatment of the at least one crosslinked polysaccharide is carried out without heating, at room temperature.

In one embodiment, this chemical treatment of the at least one crosslinked polysaccharide is carried out with heating.

In one embodiment, this chemical treatment of the at least one crosslinked polysaccharide is carried out at low temperature, using a cooling system.

In one embodiment, step e) of breaking glycosidic bonds implemented on at least one crosslinked polysaccharide is carried out by means of treatment with at least one enzyme.

In one embodiment, step e) of breaking glycosidic bonds implemented on at least one crosslinked polysaccharide is carried out by means of treatment with a mixture of enzymes.

In one embodiment, step e) of breaking glycosidic bonds implemented on at least one crosslinked polysaccharide is carried out by means of treatment with at least one hydrolase type enzyme.

In one embodiment, step e) of breaking glycosidic bonds implemented on at least one crosslinked polysaccharide is carried out by means of treatment with at least one lyase type enzyme.

In one embodiment, the enzyme used in step e) of breaking glycosidic bonds implemented on at least one crosslinked polysaccharide is chosen from the group of hyaluronidases.

In one embodiment, the hyaluronidase enzyme used in step e) of breaking glycosidic bonds implemented on at least one crosslinked polysaccharide is chosen from the group consisting of the enzymes HYAL1 and HYAL2.

In one embodiment, the hyaluronidase enzyme used in step e) of breaking glycosidic bonds implemented on at least one crosslinked polysaccharide is HYAL1.

In one embodiment, the hyaluronidase enzyme used in step e) of breaking glycosidic bonds implemented on at least one crosslinked polysaccharide is HYAL2.

In one embodiment, the enzyme used in step e) of breaking glycosidic bonds implemented on at least one crosslinked polysaccharide is an enzyme chosen from the group of bacterial enzymes.

In one embodiment, the enzyme used in step e) of breaking glycosidic bonds implemented on at least one crosslinked polysaccharide is an enzyme chosen from the group of bacterial enzymes consisting of N-acetylhexosaminidases.

In one embodiment, the N-acetylhexosaminidase enzyme is selected from the group consisting of chondroitinase ABC (CASE) and chondroitinase AC.

In one embodiment, the N-acetylhexosaminidase enzyme is chondroitinase ABC.

In one embodiment, the N-acetylhexosaminidase enzyme is chondroitinase AC.

In one embodiment, step e) of breaking glycosidic bonds implemented on at least one crosslinked polysaccharide is carried out by means of radiation treatment.

In one embodiment, this radiation treatment consists of exposing the at least one crosslinked polysaccharide to gamma radiation.

In one embodiment, this radiation treatment consists of exposing the at least one crosslinked polysaccharide to beta radiation.

In one embodiment, this radiation treatment consists of exposing the at least one crosslinked polysaccharide to a beam of accelerated electrons (electron beam).

In one embodiment, step e) of breaking glycosidic bonds implemented on at least one crosslinked polysaccharide is carried out by means of a heat treatment.

In one embodiment, this heat treatment is a step of exposing the at least one crosslinked polysaccharide to a temperature of at least 60° C. for at least 2 hours.

In one embodiment, this heat treatment is a step of exposing the at least one crosslinked polysaccharide to moist heat.

In one embodiment, this heat treatment consisting of exposing the at least one crosslinked polysaccharide to moist heat is carried out by steam autoclaving.

In one embodiment, steam autoclaving is carried out between 120° C. and 130° C.

In one embodiment, steam autoclaving is carried out with a final time equivalent (F0) of at least 30 minutes.

In one embodiment, steam autoclaving is carried out with a final time equivalent (F0) of at least 50 minutes.

In one embodiment, steam autoclaving is carried out with a final time equivalent (F0) of at least 90 minutes.

In one embodiment, this heat treatment consists of exposing the at least one crosslinked polysaccharide to dry heat.

In one embodiment, step e) of breaking glycosidic bonds implemented on at least one crosslinked polysaccharide is carried out by means of high-pressure treatment.

In one embodiment, step e) of breaking glycosidic bonds is implemented on at least one purified crosslinked polysaccharide.

In one embodiment, step e) of breaking glycosidic bonds is implemented on at least one non-purified crosslinked polysaccharide.

In one embodiment, the process for preparing the compositions according to the invention is characterised in that after step e), a step of purification of the macromolecular compound obtained is carried out.

In one embodiment, the step of purifying the macromolecular compound is a step of eliminating said crosslinking agent.

In one embodiment, the process for preparing the compositions according to the invention is characterised in that after step e), a purification step is carried out by dialysis.

In one embodiment, the process for preparing the compositions according to the invention is characterised in that after step e), a step of purifying the macromolecular compound solution is carried out by dialysis using a solution or a dialysis solvent chosen from the group consisting of phosphate buffers, for example PBS, and water.

In one embodiment, the process for preparing the compositions according to the invention is characterised in that after step e), a step of purifying the macromolecular compound solution is carried out by dialysis using a solution or a dialysis solvent chosen from the group consisting of phosphate buffers, for example PBS, further comprising at least one active ingredient, for example an antioxidant and/or a local anaesthetic, and water.

In one embodiment, the process for preparing the compositions according to the invention is characterised in that after step e), a step of purifying the macromolecular compound solution is carried out by washing.

In one embodiment, a step of purifying the macromolecular compound solution is carried out by washing using a solution or a solvent chosen from the group consisting of phosphate buffers, for example PBS, and water.

In one embodiment, a step of purifying the macromolecular compound solution is carried out by washing using a solution or a solvent chosen from the group consisting of phosphate buffers, for example PBS, further comprising at least one active ingredient, for example an antioxidant and/or a local anaesthetic, and water.

In one embodiment, the process for preparing the compositions according to the invention is characterised in that after step e), a purification step is carried out by tangential filtration.

In one embodiment, the purification step by tangential filtration comprises at least one ultrafiltration step and at least one diafiltration step.

In one embodiment, the purification step by tangential filtration comprises at least one ultrafiltration step.

In one embodiment, the purification step by tangential filtration comprises at least one diafiltration step.

In one embodiment, the composition of the macromolecular compound solution is modified during the purification step by tangential filtration.

In one embodiment, the composition of the macromolecular compound solution is concentrated during the purification step by tangential filtration.

In one embodiment, the process for preparing the compositions according to the invention is characterised in that after step e), a purification step is carried out by filtration on at least one hollow fibre membrane.

In one embodiment, the process for preparing the compositions according to the invention is characterised in that after step e), a step of purification of the macromolecular compound is carried out by precipitation in salified form.

In one embodiment, the process for preparing the compositions according to the invention is characterised in that after step e), a step of purification of the macromolecular compound is carried out by precipitation from a solution obtained after addition of PBS or NaCl salt.

In one embodiment, the process for preparing the compositions according to the invention is characterised in that after step e), a step of purification of the macromolecular compound is carried out by precipitation from a solution obtained after addition of PBS or NaCl salt, further comprising at least one active ingredient, for example an antioxidant and/or a local anaesthetic.

In one embodiment, the step of purifying the macromolecular compound by precipitation is carried out by adding at least one organic solvent to the macromolecular compound solution.

In one embodiment, the at least one organic solvent added to the macromolecular compound solution in the purification step by precipitation is chosen from the group of alcohols.

In one embodiment, ethanol is added to the macromolecular compound solution in the purification step by precipitation.

In one embodiment, the step of purifying the macromolecular compound by precipitation takes place in a hydroalcoholic mixture.

In one embodiment, the precipitated macromolecular compound is recovered by filtration.

In one embodiment, the macromolecular compound precipitated and recovered by filtration is washed.

In one embodiment, the macromolecular compound precipitated and recovered by filtration is dried.

In one embodiment, the macromolecular compound precipitated and recovered by filtration is dried at ambient atmosphere.

In one embodiment, the macromolecular compound precipitated and recovered by filtration is dried under vacuum.

The process for preparing the compositions according to the invention further comprises at least one step of filtration on a membrane with a porosity of 0.22 μm, step g), of the aqueous macromolecular compound solution resulting from step e), this step of filtration being carried out on the aqueous macromolecular compound solution or on a sample of the aqueous solution of macromolecular compound.

In one embodiment, this filtration step on a membrane with a porosity of 0.22 μm is the terminal sterilization step.

In one embodiment, this filtration step on a membrane with a porosity of 0.22 μm is carried out once.

In one embodiment, this filtration step on a membrane with a porosity of 0.22 μm is carried out twice.

In one embodiment, this filtration step on a membrane with a porosity of 0.22 μm is carried out three times.

In one embodiment, the filtration step on a membrane with a porosity of 0.22 μm is a means of monitoring the process in progress or “in-process control” (IPC) and is carried out on a sample of the aqueous macromolecular compound solution obtained after step e) of the process.

In one embodiment, the filtration IPC on a membrane with a porosity of 0.22 μm is carried out on a sample of the aqueous macromolecular compound solution obtained after step e) of the process after a sample dilution step.

In one embodiment, if step e) of breaking the glycosidic bonds is carried out by steam autoclaving, then the process for preparing the composition according to the invention does not include a filtration step on a membrane of porosity 0.22 μm of the macromolecular compound solution.

In one embodiment, if step e) of breaking the glycosidic bonds is carried out by steam autoclaving, then the process of preparing the composition comprises a step of taking a sample of the aqueous macromolecular compound solution after step e) and filterability test of the sample on a membrane with a porosity of 0.22 μm (IPC).

In one embodiment, the composition according to the invention is characterised by G′ less than or equal to 300 Pa at 1 Hz.

In one embodiment, the composition according to the invention is characterised by G′ less than or equal to 150 Pa at 1 Hz.

In one embodiment, the composition according to the invention is characterised by G′ less than or equal to 100 Pa at 1 Hz.

In one embodiment, the composition according to the invention is characterised by G′ less than or equal to 85 Pa at 1 Hz.

In one embodiment, the composition according to the invention is characterised by G′ less than or equal to 70 Pa at 1 Hz.

In one embodiment, the composition according to the invention is characterised by Tan Δ (Tn δ) greater than or equal to 1.00.

In one embodiment, the composition according to the invention is characterised by Tan Δ (Tn δ) between 1.00 and 1.80 (1.00≤Tan Δ (Tn δ)≤1.80).

In one embodiment, the composition according to the invention is characterised by Tan Δ (Tn δ) between 1.00 and 1.50 (1.00≤Tan Δ (Tn δ)≤1.50).

In one embodiment, the composition according to the invention is characterised by Tan Δ (Tn δ) between 1.00 and 1.30 (1.00≤Tan Δ (Tn δ)≤1.30).

The invention also relates to a process for preparing a formulation comprising the composition according to the invention.

In one embodiment, the process for preparing a formulation from the composition of the invention further comprises at least one dilution or dissolution step.

In one embodiment, the dilution or dissolution step is carried out by adding water or an aqueous saline solution, for example a phosphate buffer solution, for example PBS.

In one embodiment, the dilution or dissolution step is carried out by adding water or an aqueous saline solution, for example a phosphate buffer solution, for example PBS, further comprising at least one active ingredient, for example an antioxidant and/or a local anaesthetic.

In one embodiment, the process for preparing a formulation comprising the composition according to the invention further comprises at least one dilution or dissolution step to obtain a macromolecular compound concentration of between 2 mg/g and 200 mg/g, relative to the total mass of said formulation.

In one embodiment, the process for preparing a formulation comprising the composition according to the invention further comprises at least one dilution or dissolution step to obtain a macromolecular compound concentration of between 2 mg/g and 75 mg/g, relative to the total mass of said formulation.

In one embodiment, the process for preparing a formulation comprising the composition according to the invention further comprises at least one dilution or dissolution step to obtain a macromolecular compound concentration of between 5 mg/g and 50 mg/g, relative to the total mass of said formulation.

In one embodiment, the process for preparing a formulation comprising the composition according to the invention further comprises at least one dilution or dissolution step to obtain a macromolecular compound concentration of between 10 mg/g and 40 mg/g, relative to the total mass of said formulation.

In one embodiment, the process for preparing a formulation comprising the composition according to the invention further comprises at least one dilution or dissolution step to obtain a macromolecular compound concentration of approximately 100 mg/g, relative to the total mass of said formulation.

In one embodiment, the process for preparing a formulation comprising the composition according to the invention further comprises at least one dilution or dissolution step to obtain a macromolecular compound concentration of approximately 80 mg/g, relative to the total mass of said formulation.

In one embodiment, the process for preparing a formulation comprising the composition according to the invention further comprises at least one dilution or dissolution step to obtain a macromolecular compound concentration of approximately 60 mg/g, relative to the total mass of said formulation.

In one embodiment, the process for preparing a formulation comprising the composition according to the invention further comprises at least one dilution or dissolution step to obtain a macromolecular compound concentration of approximately 40 mg/g, relative to the total mass of said formulation.

In one embodiment, the process for preparing a formulation comprising the composition according to the invention further comprises at least one dilution or dissolution step to obtain a macromolecular compound concentration of approximately 20 mg/g, relative to the total mass of said formulation.

In one embodiment, the process for preparing a formulation comprising the composition according to the invention further comprises at least one dilution or dissolution step to obtain a macromolecular compound concentration of approximately 10 mg/g, relative to the total mass of said formulation.

In one embodiment, the process for preparing the compositions according to the invention further comprises at least one step of adding at least one active ingredient.

In one embodiment, the at least one active ingredient is added in powder form.

In one embodiment, the at least one active ingredient is added in the form of a solution or suspension.

In one embodiment, the at least one active ingredient is added in the form of a solution or suspension, in a solvent or a solution chosen from the group consisting of water, aqueous saline solutions, for example a phosphate buffer solution, such as PBS.

In one embodiment, the at least one active ingredient is added before step e) of the process for preparing the compositions according to the invention.

In one embodiment, the at least one active ingredient is added after step e) of the process for preparing the compositions according to the invention.

In one embodiment, the at least one active ingredient is added before step g) of the process of the compositions according to the invention.

In one embodiment, the at least one active ingredient is added after step g) of the process of the compositions according to the invention.

In one embodiment, the formulation obtained by adding at least one active ingredient after step g) of the process for preparing the compositions according to the invention is sterilised by filtration on a membrane with a porosity of 0.22 μm.

In one embodiment, the process for preparing a formulation comprising at least one composition according to the invention further comprises at least one step of adding at least one active ingredient chosen from the group consisting of local anaesthetics, derivatives vitamin C, anti-inflammatory substances, antioxidants, and mixtures thereof.

In one embodiment, the process for preparing a formulation comprising at least one composition according to the invention further comprises at least one step of adding at least one local anaesthetic.

In one embodiment, the process for preparing a formulation comprising at least one composition according to the invention further comprises at least one step of adding at least one local anaesthetic to obtain a local anaesthetic concentration of between 0.1 and 5%, relative to the total mass of said formulation.

In one embodiment, the process for preparing a formulation comprising at least one composition according to the invention further comprises at least one step of adding at least one local anaesthetic to obtain a local anaesthetic concentration of between 0.1 and 4%, relative to the total mass of said formulation.

In one embodiment, the process for preparing a formulation comprising at least one composition according to the invention further comprises at least one step of adding at least one local anaesthetic to obtain a local anaesthetic concentration of between 0.1 and 2%, relative to the total mass of said formulation.

In one embodiment, the process for preparing a formulation comprising at least one composition according to the invention further comprises at least one step of adding at least one local anaesthetic to obtain a local anaesthetic concentration of between 0.1 and 1%, relative to the total mass of said formulation.

In one embodiment, the process for preparing a formulation comprising at least one composition according to the invention further comprises at least one step of adding at least one local anaesthetic to obtain a local anaesthetic concentration of between 0.1 and 0.5%, relative to the total mass of said formulation.

In one embodiment, the process for preparing a formulation comprising at least one composition according to the invention further comprises at least one step of adding at least one local anaesthetic to obtain a local anaesthetic concentration of approximately 0.3%, relative to the total mass of said formulation.

In one embodiment, the local anaesthetic is chosen from the group of amino esters.

In one embodiment, the amino ester is chosen from the group comprising procaine, benzocaine, chloroprocaine and tetracaine in base or salt form, for example in hydrochloride form.

In one embodiment, the local anaesthetic is chosen from the group of aminoamides.

In one embodiment, the amino amide is chosen from the group comprising lidocaine, mepivacaine, prilocaine, articaine, aptocaine, bupivacaine, etidocaine and ropivacaine in base or salt form, for example in the form of hydrochloride.

In one embodiment, the local anaesthetic is chosen from the group of amino ethers.

In one embodiment, the amino ether is chosen from the group comprising diamocaine and pramocaine in base or salt form, for example in hydrochloride or cyclamate form.

In one embodiment, the amino ether is chosen from the group consisting of lidocaine, mepivacaine, and their salts and their isolated isomers.

In one embodiment, the amino ether is lidocaine.

In one embodiment, the amino ether is lidocaine or a pharmaceutically acceptable salt thereof.

In one embodiment, the amino ether is lidocaine hydrochloride.

In one embodiment, the amino ether is mepivacaine.

In one embodiment, the amino ether is mepivacaine or a pharmaceutically acceptable salt thereof.

In one embodiment, the amino ether is chosen from the group consisting of racemic mepivacaine hydrochloride, (r)-mepivacaine hydrochloride, (s)-mepivacaine hydrochloride, (r)-mepivacaine and(s)-mepivacaine, or one of their pharmaceutically acceptable salts.

In one embodiment, the amino ether is mepivacaine hydrochloride.

In one embodiment, the amino ether is (r)-mepivacaine hydrochloride.

In one embodiment, the amino ether is (s)-mepivacaine hydrochloride.

In one embodiment, the amino ether is racemic mepivacaine hydrochloride.

In one embodiment, the amino ether is (r)-mepivacaine.

In one embodiment, the amino ether is (s)-mepivacaine.

In one embodiment, the amino ether is racemic mepivacaine.

In one embodiment, the process for preparing a formulation comprising at least one composition according to the invention further comprises at least one step of adding at least one amino-ether local anaesthetic chosen from the group consisting of lidocaine, mepivacaine, and mixtures thereof.

In one embodiment, the process for preparing a formulation comprising at least one composition according to the invention further comprises at least one step of adding at least one local anaesthetic being lidocaine.

In one embodiment, the process for preparing a formulation comprising at least one composition according to the invention further comprises at least one step of adding at least one local anaesthetic being lidocaine to obtain a lidocaine concentration comprised between 0.1 and 5%, relative to the total mass of said formulation.

In one embodiment, the process for preparing a formulation comprising at least one composition according to the invention further comprises at least one step of adding at least one local anaesthetic being lidocaine to obtain a lidocaine concentration comprised between 0.1 and 4%, relative to the total mass of said formulation.

In one embodiment, the process for preparing a formulation comprising at least one composition according to the invention further comprises at least one step of adding at least one local anaesthetic being lidocaine to obtain a lidocaine concentration comprised between 0.1 and 2%, relative to the total mass of said formulation.

In one embodiment, the process for preparing a formulation comprising at least one composition according to the invention further comprises at least one step of adding at least one local anaesthetic being lidocaine to obtain a lidocaine concentration comprised between 0.1 and 1%, relative to the total mass of said formulation.

In one embodiment, the process for preparing a formulation comprising at least one composition according to the invention further comprises at least one step of adding at least one local anaesthetic being lidocaine to obtain a lidocaine concentration comprised between 0.1 and 0.5%, relative to the total mass of said formulation.

In one embodiment, the process for preparing a formulation comprising at least one composition according to the invention further comprises at least one step of adding at least one local anaesthetic being lidocaine to obtain a concentration of local anaesthetic approximately 0.3%, relative to the total mass of said formulation.

In one embodiment, the process for preparing a formulation comprising at least one composition according to the invention further comprises at least one step of adding at least one local anaesthetic being mepivacaine.

In one embodiment, the process for preparing a formulation comprising at least one composition according to the invention further comprises at least one step of adding at least one local anaesthetic being mepivacaine to obtain a concentration of mepivacaine comprised between 0.1 and 5%, relative to the total mass of said formulation.

In one embodiment, the process for preparing a formulation comprising at least one composition according to the invention further comprises at least one step of adding at least one local anaesthetic being mepivacaine to obtain a concentration of mepivacaine comprised between 0.1 and 4%, relative to the total mass of said formulation.

In one embodiment, the process for preparing a formulation comprising at least one composition according to the invention further comprises at least one step of adding at least one local anaesthetic being mepivacaine to obtain a concentration of mepivacaine comprised between 0.1 and 2%, relative to the total mass of said formulation.

In one embodiment, the process for preparing a formulation comprising at least one composition according to the invention further comprises at least one step of adding at least one local anaesthetic being mepivacaine to obtain a concentration of mepivacaine comprised between 0.1 and 1%, relative to the total mass of said formulation.

In one embodiment, the process for preparing a formulation comprising at least one composition according to the invention further comprises at least one step of adding at least one local anaesthetic being mepivacaine to obtain a concentration of mepivacaine comprised between 0.1 and 0.5%, relative to the total mass of said formulation.

In one embodiment, the process for preparing a formulation comprising at least one composition according to the invention further comprises at least one step of adding at least one local anaesthetic being mepivacaine to obtain a concentration of local anaesthetic approximately 0.3%, relative to the total mass of said formulation.

In one embodiment, the process for preparing a formulation comprising at least one composition according to the invention further comprises at least one step of adding at least one local anaesthetic being dyclonine in base or salt form, for example in the form of hydrochloride.

In one embodiment, the process for preparing a formulation comprising at least one composition according to the invention further comprises at least one step of adding at least one local anaesthetic chosen from the group consisting of chlorobutanol, guafecainol and polidocanol.

In one embodiment, the local anaesthetic is chlorobutanol.

In one embodiment, the local anaesthetic is guafecainol.

In one embodiment, the local anaesthetic is polidocanol.

In one embodiment, the process for preparing a formulation comprising at least one composition according to the invention further comprises at least one step of adding at least one anti-inflammatory substance.

In one embodiment, the process for preparing a formulation comprising at least one composition according to the invention further comprises at least one step of adding at least one anti-inflammatory substance chosen from the group consisting of steroidal and non-steroidal anti-inflammatory substances.

In one embodiment, the process for preparing a formulation comprising at least one composition according to the invention further comprises at least one step of adding at least one anti-inflammatory substance chosen from the group consisting of non-steroidal anti-inflammatory substances.

In one embodiment, the process for preparing a formulation comprising at least one composition according to the invention further comprises at least one step of adding at least one anti-inflammatory substance chosen from the group comprising salicylated anti-inflammatory substances, propionic derivatives, indole derivatives, pyrazole derivatives, oxicams and coxibs.

In one embodiment, the process for preparing a formulation comprising at least one composition according to the invention further comprises at least one step of adding at least one anti-inflammatory substance chosen from the group comprising diclofenac, nimesulfide, niflumic acid, mefenamic acid and nabumetone, alone or in a mixture.

In one embodiment, the process for preparing a formulation comprising at least one composition according to the invention further comprises at least one step of adding at least one salicylated anti-inflammatory substance chosen from the group comprising diflunisal, benorilate and aspirin, alone or in a mixture.

In one embodiment, the process for preparing a formulation comprising at least one composition according to the invention further comprises at least one step of adding at least one anti-inflammatory substance chosen from the group of propionic derivatives comprising alminoprofen, ketoprofen, ibuprofen, naproxen, flurbiprofen and tiaprofenic acid, alone or in a mixture.

In one embodiment, the process for preparing a formulation comprising at least one composition according to the invention further comprises at least one step of adding at least one anti-inflammatory substance chosen from the group of indole derivatives including indomethacin, sulindac and etodolac, alone or in a mixture.

In one embodiment, the process for preparing a formulation comprising at least one composition according to the invention further comprises at least one step of adding at least one anti-inflammatory substance chosen from the group of pyrazole derivatives including in particular phenylbutazone.

In one embodiment, the process for preparing a formulation comprising at least one composition according to the invention further comprises at least one step of adding at least one anti-inflammatory substance chosen from the group of oxicams including piroxicam, tenoxicam and meloxicam, alone or in a mixture.

In one embodiment, the process for preparing a formulation comprising at least one composition according to the invention further comprises at least one step of adding at least one anti-inflammatory substance chosen from the group of coxibs including celecoxib, etoricoxib and rofecoxib, alone or in a mixture.

In one embodiment, the concentration of non-steroidal anti-inflammatory substances in a formulation comprising at least one composition according to the invention is between 0.01 and 2000 mg/g.

In one embodiment, the concentration of non-steroidal anti-inflammatory substances in a formulation comprising at least one composition according to the invention is between 0.1 and 1000 mg/g.

In one embodiment, the concentration of non-steroidal anti-inflammatory substances in a formulation comprising at least one composition according to the invention is between 0.5 and 500 mg/g.

In one embodiment, the process for preparing a formulation comprising at least one composition according to the invention further comprises at least one step of adding at least one steroidal anti-inflammatory substance.

In one embodiment, the process for preparing a formulation comprising at least one composition according to the invention further comprises at least one step of adding at least one steroidal anti-inflammatory substance chosen from the group comprising dexamethasone, prednisolone, corticosterone, budesonide, sulfasalazine, mesalamine, cetirizine, diphenhydramine, antipyrine, methyl salicylate, loratadine, thymol, carvacrol, bisabolol, allantoin, eucalyptol, phenazone (antipyrine), propyphenazone, alone or in a mixture.

In one embodiment, the concentration of steroidal anti-inflammatory substances in a formulation comprising at least one composition according to the invention is between 0.01 and 2000 mg/g.

In one embodiment, the concentration of steroidal anti-inflammatory substances in a formulation comprising at least one composition according to the invention is between 0.1 and 1000 mg/g.

In one embodiment, the concentration of steroidal anti-inflammatory substances in a formulation comprising at least one composition according to the invention is between 0.5 and 500 mg/g.

In one embodiment, the process for preparing a formulation comprising at least one composition according to the invention further comprises at least one step of adding at least one anti-inflammatory substance chosen from the group consisting of sucrose octasulfate and its salts.

In one embodiment, the process for preparing a formulation comprising at least one composition according to the invention further comprises at least one step of adding at least one anti-inflammatory substance chosen from the group consisting of sucrose octasulfate and its sodium and potassium salts.

In one embodiment, the process for preparing a formulation comprising at least one composition according to the invention further comprises at least one step of adding at least one anti-inflammatory substance water-soluble salt of sucrose octasulfate chosen from the group consisting of alkali metal salts, alkaline-earth metal salts, silver salts, ammonium salts, amino acid salts.

In one embodiment, the process for preparing a formulation comprising at least one composition according to the invention further comprises at least one step of adding at least one water-soluble anti-inflammatory salt of sucrose octasulfate chosen from the group consisting of alkali metal salts or alkaline-earth metal salts.

In one embodiment, the process for preparing a formulation comprising at least one composition according to the invention further comprises at least one step of adding at least one water-soluble anti-inflammatory salt of sucrose octasulfate chosen from the group consisting of the sodium salt of sucrose octasulfate or the potassium salt of sucrose octasulfate.

In one embodiment, the process for preparing a formulation comprising at least one composition according to the invention further comprises at least one step of adding at least one antimicrobial substance.

In one embodiment, the process for preparing a formulation comprising at least one composition according to the invention further comprises at least one step of adding at least one antimicrobial substance chosen from the group comprising gentamicin, silver sulfadiazine, metronidazole, fucidine, bacitracin, eosin, povidone-lodine, copper gluconate, zinc gluconate, manganese gluconate or their salts, alone or in a mixture.

In one embodiment, the concentration of antimicrobial substances in a formulation comprising at least one composition according to the invention is between 0.1 and 200 mg/g.

In one embodiment, the concentration of antimicrobial substances in a formulation comprising at least one composition according to the invention is between 0.5 and 100 mg/g.

In one embodiment, the process for preparing a formulation comprising at least one composition according to the invention further comprises at least one step of adding at least one glycoside or a glycoside derivative.

In one embodiment, the process for preparing a formulation comprising at least one composition according to the invention further comprises at least one step of adding at least one glycoside or a glycoside derivative chosen from the group comprising D-glucopyranose, 1,4 glycoside, esculin, hesperidin, diosmin, arbutin, skimmin or aloin, alone or in mixtures.

In one embodiment, the concentration of glycosides in a formulation comprising at least one composition according to the invention is between 0.1 and 200 mg/g.

In one embodiment, the concentration of glycosides in a formulation comprising at least one composition according to the invention is between 0.5 and 100 mg/g.

In one embodiment, the process for preparing a formulation comprising at least one composition according to the invention further comprises at least one step of adding at least one moisturizer or tissue regenerator.

In one embodiment, the process for preparing a formulation comprising at least one composition according to the invention further comprises at least one step of adding at least one moisturizer or tissue regenerator chosen from the group consisting of macro-elements.

In one embodiment, the process for preparing a formulation comprising at least one composition according to the invention further comprises at least one step of adding at least one macro-element chosen from the group comprising iron, calcium, copper, zinc, manganese, magnesium or potassium gluconates, timethylsilanol, trimethylsilanolate, potassium trimethylsilanolate, methylsilanol mannuronate, sodium monoethyltrisilanol orthohydroxybenzoate, alone or in a mixture.

In one embodiment, the process for preparing a formulation comprising at least one composition according to the invention further comprises at least one step of adding at least one moisturizer or tissue regenerator chosen from the group consisting of essential amino acids.

In one embodiment, the process for preparing a formulation comprising at least one composition according to the invention further comprises at least one step of adding at least one essential amino acid chosen from the group comprising isoleucine, leucine, lysine, methionine, phenylalanine, threonine, tryptophan and valine, alone or in a mixture.

In one embodiment, the process for preparing a formulation comprising at least one composition according to the invention further comprises at least one step of adding at least one moisturizer or tissue regenerator chosen from the group consisting of amino acids semi-essential.

In one embodiment, the process for preparing a formulation comprising at least one composition according to the invention further comprises at least one step of adding at least one semi-essential amino acid chosen from the group comprising arginine and histidine, alone or in a mixture.

In one embodiment, the process for preparing a formulation comprising at least one composition according to the invention further comprises at least one step of adding at least one moisturizer or tissue regenerator chosen from the group consisting of amino acids non-essential.

In one embodiment, the process for preparing a formulation comprising at least one composition according to the invention further comprises at least one step of adding at least one non-essential amino acid chosen from the group comprising alanine, asparagine, aspartic acid, cysteine, glutamine, glutamic acid, glycine, proline, serine, tyrosine, alone or in a mixture.

In one embodiment, the process for preparing a formulation comprising at least one composition according to the invention further comprises at least one step of adding at least one moisturizer or tissue regenerator chosen from the group consisting of vitamins.

In one embodiment, the process for preparing a formulation comprising at least one composition according to the invention further comprises at least one step of adding at least one vitamin chosen from the group consisting of retinol, thiamine, riboflavin, nicotinamide, adenine, calcium pantothenate, pyridoxine, inositol, biotin, folic acid, para-amino-benzoic acid, cobalamin, vitamin C, choline chloride, alone or in a mixture.

In one embodiment, the process for preparing a formulation comprising at least one composition according to the invention further comprises at least one step of adding at least one moisturizer or tissue regenerator chosen from the group consisting of nucleic acids.

In one embodiment, the process for preparing a formulation comprising at least one composition according to the invention further comprises at least one step of adding at least one nucleic acid chosen from the group consisting of deoxyadenosine, deoxycytidine, deoxyguanosine, deoxythymidine, methylcytosine, alone or in a mixture.

In one embodiment, the process for preparing a formulation comprising at least one composition according to the invention further comprises at least one step of adding at least one moisturizer or tissue regenerator chosen from the group consisting of coenzymes.

In one embodiment, the process for preparing a formulation comprising at least one composition according to the invention further comprises at least one step of adding at least one coenzyme chosen from the group consisting of thiamine pyrophosphate, coenzyme A, FAD, NAD, NADP, UTP, alone or in a mixture.

In one embodiment, the process for preparing a formulation comprising at least one composition according to the invention further comprises at least one step of adding at least one moisturizer or tissue regenerator chosen from the group consisting of deoxythymidine, glutathione, sodium pyruvate, lipoic acid and putrescine, alone or in a mixture.

In one embodiment, the concentration of moisturizers or tissue regenerators in a formulation comprising at least one composition according to the invention is between 0.01 and 500 mg/g.

In one embodiment, the concentration of moisturizers or tissue regenerators in a formulation comprising at least one composition according to the invention is between 0.1 and 200 mg/g.

In one embodiment, the process for preparing a formulation comprising at least one composition according to the invention further comprises at least one step of adding at least one antioxidant.

In one embodiment, the process for preparing a formulation comprising at least one composition according to the invention further comprises at least one step of adding at least one antioxidant chosen from the group of polyols.

In one embodiment, the process for preparing a formulation comprising at least one composition according to the invention further comprises at least one step of adding at least one polyol chosen from the group consisting of mannitol, sorbitol, propylene glycol, xylitol, glycerol, maltitol, lactitol and erythritol.

In one embodiment, the process for preparing a formulation comprising at least one composition according to the invention further comprises at least one step of adding at least one polyol chosen from the group consisting of mannitol, sorbitol, maltitol and glycerol, alone or in a mixture.

In one embodiment, the process for preparing a formulation comprising at least one composition according to the invention further comprises at least one step of adding at least one polyol chosen from the group consisting of mannitol, sorbitol and maltitol, alone or in a mixture.

In one embodiment, the process for preparing a formulation comprising at least one composition according to the invention further comprises at least one step of adding at least one polyol to obtain a polyol concentration of between 0.1 mg/ml and 50 mg/ml, relative to the total mass of said formulation.

In one embodiment, the process for preparing a formulation comprising at least one composition according to the invention further comprises at least one step of adding at least one polyol to obtain a polyol concentration of between 5 mg/ml and 40 mg/ml, relative to the total mass of said formulation.

In one embodiment, the process for preparing a formulation comprising at least one composition according to the invention further comprises at least one step of adding at least one polyol to obtain a polyol concentration of between 10 mg/ml and 40 mg/ml, relative to the total mass of said formulation.

In one embodiment, the process for preparing a formulation comprising at least one composition according to the invention further comprises at least one step of adding at least one polyol to obtain a polyol concentration of between 20 mg/ml and 40 mg/ml, relative to the total mass of said formulation.

In one embodiment, the process for preparing a formulation comprising at least one composition according to the invention further comprises at least one step of adding at least one polyol to obtain a polyol concentration of between 30 mg/ml and 40 mg/ml, relative to the total mass of said formulation.

In one embodiment, the process for preparing a formulation comprising at least one composition according to the invention further comprises at least one step of adding at least one polyol being mannitol.

In one embodiment, the process for preparing a formulation comprising at least one composition according to the invention further comprises at least one step of adding mannitol to obtain a mannitol concentration of between 5 mg/ml and 40 mg/ml, relative to the total mass of said formulation.

In one embodiment, the process for preparing a formulation comprising at least one composition according to the invention further comprises at least one step of adding mannitol to obtain a mannitol concentration of between 10 mg/ml and 40 mg/ml, relative to the total mass of said formulation.

In one embodiment, the process for preparing a formulation comprising at least one composition according to the invention further comprises at least one step of adding mannitol to obtain a mannitol concentration of between 20 mg/ml and 40 mg/ml, relative to the total mass of said formulation.

In one embodiment, the process for preparing a formulation comprising at least one composition according to the invention further comprises at least one step of adding mannitol to obtain a mannitol concentration of between 30 mg/ml and 40 mg/ml, relative to the total mass of said formulation.

In one embodiment, the process for preparing a formulation comprising at least one composition according to the invention further comprises at least one step of adding at least one polyol being sorbitol.

In one embodiment, the process for preparing a formulation comprising at least one composition according to the invention further comprises at least one step of adding sorbitol to obtain a sorbitol concentration of between 5 mg/ml and 40 mg/ml, relative to the total mass of said formulation.

In one embodiment, the process for preparing a formulation comprising at least one composition according to the invention further comprises at least one step of adding sorbitol to obtain a sorbitol concentration of between 10 mg/ml and 40 mg/ml, relative to the total mass of said formulation.

In one embodiment, the process for preparing a formulation comprising at least one composition according to the invention further comprises at least one step of adding sorbitol to obtain a sorbitol concentration of between 20 mg/ml and 40 mg/ml, relative to the total mass of said formulation.

In one embodiment, the process for preparing a formulation comprising at least one composition according to the invention further comprises at least one step of adding sorbitol to obtain a sorbitol concentration of between 30 mg/ml and 40 mg/ml, relative to the total mass of said formulation.

In one embodiment, the process for preparing a formulation comprising at least one composition according to the invention further comprises at least one step of adding at least one polyol being maltitol.

In one embodiment, the process for preparing a formulation comprising at least one composition according to the invention further comprises at least one step of adding at least one polyol being glycerol.

In one embodiment, the process for preparing a formulation comprising at least one composition according to the invention further comprises at least one step of adding a mixture of mannitol and sorbitol.

In one embodiment, the process for preparing a formulation comprising at least one composition according to the invention further comprises at least one step of adding at least one antioxidant chosen from the group of vitamin C derivatives.

In one embodiment, the process for preparing a formulation comprising at least one composition according to the invention further comprises at least one step of adding at least one antioxidant chosen from the group of vitamin C derivatives comprising magnesium ascorbyl phosphate, sodium ascorbyl phosphate, ascorbyl-2-glucoside, and their mixture.

In one embodiment, said at least one vitamin C derivative is magnesium ascorbyl phosphate.

In one embodiment, the process for preparing a formulation comprising at least one composition according to the invention further comprises at least one step of adding at least one antioxidant chosen from the group of vitamin E derivatives and tocopherols.

In one embodiment, the process for preparing a formulation comprising at least one composition according to the invention further comprises at least one step of adding at least one antioxidant chosen from the group of carotenoids and retinoids and their derivatives.

In one embodiment, the process for preparing a formulation comprising at least one composition according to the invention further comprises at least one step of adding at least one antioxidant chosen from the group of carotenoids and retinoids and their derivatives including retinol, retinoic acid, retinal, retinol esters and carotene.

In one embodiment, the process for preparing a formulation comprising at least one composition according to the invention further comprises at least one step of adding at least one antioxidant chosen from the group of pseudo-tripeptides. In one embodiment, the pseudo-tripeptide is glutathione.

In one embodiment, the process for preparing a formulation comprising at least one composition according to the invention further comprises at least one step of adding at least one antioxidant chosen from the group comprising the different forms of the coenzyme Q10, ubiquinone or ubiquinol.

In one embodiment, the process for preparing a formulation comprising at least one composition according to the invention further comprises at least one step of adding at least one vitamin.

In one embodiment, the process for preparing a formulation comprising at least one composition according to the invention further comprises at least one step of adding at least one vitamin chosen from the group comprising retinol, thiamine, riboflavin, nicotinamide, dexpenthenol, piridoxine, ascorbic acid, ergocalciferol, tocopherol, biotin and folic acid alone or in a mixture.

In one embodiment, the vitamin concentration in a formulation comprising at least one composition according to the invention is between 0.01 and 200 mg/g.

In one embodiment, the vitamin concentration in a formulation comprising at least one composition according to the invention is between 0.01 and 100 mg/g.

In one embodiment, the concentration of vitamins in a formulation comprising at least one composition according to the invention is between 0.5 and 50 mg/g.

In one embodiment, the process for preparing a formulation comprising at least one composition according to the invention further comprises at least one step of adding at least one amino acid.

In one embodiment, the process for preparing a formulation comprising at least one composition according to the invention further comprises at least one step of adding at least one amino acid chosen from the group consisting of essential amino acids, semi-essential amino acids and non-essential amino acids, alone or in a mixture.

In one embodiment, the process for preparing a formulation comprising at least one composition according to the invention further comprises at least one step of adding at least one amino acid chosen from the group of essential amino acids.

In one embodiment, the process for preparing a formulation comprising at least one composition according to the invention further comprises at least one step of adding at least one essential amino acid chosen from the group comprising isoleucine, leucine, lysine, methionine, phenylalanine, threonine, tryptophan and valine, alone or in a mixture.

In one embodiment, the process for preparing a formulation comprising at least one composition according to the invention further comprises at least one step of adding at least one amino acid chosen from the group of semi-essential amino acids.

In one embodiment, the process for preparing a formulation comprising at least one composition according to the invention further comprises at least one step of adding at least one semi-essential amino acid chosen from the group comprising arginine and histidine, alone or in a mixture.

In one embodiment, the process for preparing a formulation comprising at least one composition according to the invention further comprises at least one step of adding at least one amino acid chosen from the group of non-essential amino acids.

In one embodiment, the process for preparing a formulation comprising at least one composition according to the invention further comprises at least one step of adding at least one non-essential amino acid chosen from the group comprising alanine, asparagine, aspartic acid, cysteine, glutamine, glutamic acid, glycine, proline, serine, tyrosine, alone or in a mixture.

In one embodiment, the process for preparing a formulation comprising at least one composition according to the invention further comprises at least one step of adding at least one amino acid chosen from the group comprising hydroxyproline, taurine and ornithine, alone or in a mixture.

In one embodiment, the concentration of amino acids in a formulation comprising at least one composition according to the invention is between 0.01 and 150 mg/g.

In one embodiment, the concentration of amino acids in a formulation comprising at least one composition according to the invention is between 0.01 and 100 mg/g.

In one embodiment, the concentration of amino acids in a formulation comprising at least one composition according to the invention is between 0.5 and 50 mg/g.

In one embodiment, the process for preparing a formulation comprising at least one composition according to the invention further comprises at least one step of adding at least one vasoconstrictor.

In one embodiment, the process for preparing a formulation comprising at least one composition according to the invention further comprises at least one step of adding at least one vasoconstrictor chosen from the group comprising naphazoline, epinephrine, methoxamine, methylnorepinephrine, norepinephrine, oxymethazoline, phenylephrine, pseudoephedrine, synephrine, cirazoline and xylomethazoline.

In one embodiment, the concentration of vasoconstrictors in a formulation comprising at least one composition according to the invention is between 0.01 and 3 mg/g.

In one embodiment, the process for preparing a formulation comprising at least one composition according to the invention further comprises at least one step of adding at least one vasodilator.

In one embodiment, the process for preparing a formulation comprising at least one composition according to the invention further comprises at least one step of adding at least one vasodilator chosen from the group comprising adenosine, nicotinic acid, minoxidil and diazoxide, alone or in a mixture.

In one embodiment, the concentration of vasodilators in a formulation comprising at least one composition according to the invention is between 0.01 and 10 mg/g.

In one embodiment, the process for preparing a formulation comprising at least one composition according to the invention further comprises at least one step of adding at least one anti-haemorrhagic or haemostatic agent.

In one embodiment, the process for preparing a formulation comprising at least one composition according to the invention further comprises at least one step of adding at least one anti-haemorrhagic or haemostatic agent chosen from the group comprising aminocaproic acid or tranexamic acid, alone or in a mixture.

In one embodiment, the concentration of anti-haemorrhagics or haemostatics in a formulation comprising at least one composition according to the invention is between 0.01 and 5 mg/g.

In one embodiment, the process for preparing a formulation comprising at least one composition according to the invention further comprises at least one step of adding at least one antioxidant and at least one local anaesthetic.

In one embodiment, the process for preparing a formulation comprising at least one composition according to the invention further comprises at least one step of adding at least one antioxidant chosen from the group of polyols and at least one local anaesthetic chosen from the group of amino amides.

In one embodiment, the process for preparing a formulation comprising at least one composition according to the invention further comprises at least one step of adding at least one antioxidant, at least one vitamin and at least one tissue regenerator.

In one embodiment, the process for preparing a formulation comprising at least one composition according to the invention further comprises at least one step of adding at least one amino acid, at least one vitamin and at least one tissue regenerator.

In one embodiment, the process for preparing a formulation comprising at least one composition according to the invention further comprises at least one step of adding at least one antioxidant, at least one amino acid, at least one vitamin and at least one tissue regenerator.

In one embodiment, the process for preparing a formulation comprising at least one composition according to the invention further comprises at least one step of adding at least one active ingredient chosen from the group consisting of heat-sensitive active ingredients.

In one embodiment, the at least one heat-sensitive active ingredient is added after step e) of the process for preparing the at least one composition according to the invention.

In one embodiment, the process for preparing a formulation comprising at least one composition according to the invention further comprises at least one step of adding a so-called thermosensitive active ingredient of natural origin.

In one embodiment, the process for preparing a formulation comprising at least one composition according to the invention further comprises at least one step of adding a so-called thermosensitive active ingredient of synthetic origin.

In one embodiment, the process for preparing a formulation comprising at least one composition according to the invention further comprises at least one step of adding at least one thermosensitive active ingredient chosen from the group consisting of peptides, hormones, proteins, growth factors, antibodies and vitamins, alone or in mixtures.

In one embodiment, the process for preparing a formulation comprising at least one composition according to the invention further comprises at least one step of adding at least one thermosensitive active ingredient chosen from the group consisting of peptides.

In one embodiment, the added thermosensitive active ingredient is a peptide.

In one embodiment, the process for preparing a formulation comprising at least one composition according to the invention further comprises at least one step of adding at least one heat-sensitive active ingredient chosen from the group consisting of hormones.

In one embodiment, the added thermosensitive active ingredient is a hormone.

In one embodiment, the process for preparing a formulation comprising at least one composition according to the invention further comprises at least one step of adding at least one thermosensitive active ingredient chosen from the group consisting of proteins.

In one embodiment, the added thermosensitive active ingredient is a protein.

In one embodiment, the process for preparing a formulation comprising at least one composition according to the invention further comprises at least one step of adding at least one thermosensitive active ingredient chosen from the group consisting of growth factors.

In one embodiment, the added thermosensitive active ingredient is a growth factor.

In one embodiment, the process for preparing a formulation comprising at least one composition according to the invention further comprises at least one step of adding at least one thermosensitive active ingredient chosen from the group consisting of antibodies.

In one embodiment, the added thermosensitive active ingredient is an antibody.

In one embodiment, the process for preparing a formulation comprising at least one composition according to the invention further comprises at least one step of adding at least one heat-sensitive active ingredient chosen from the group consisting of vitamins.

In one embodiment, the vitamins are chosen from the group comprising retinol, thiamine, riboflavin, nicotinamide, dexpenthenol, piridoxine, ascorbic acid, ergocalciferol, tocopherol, biotin and acid folic alone or in a mixture.

The invention also relates to a formulation comprising at least one composition according to the invention.

In one embodiment, the formulation is characterised in that the concentration of macromolecular compound is between 2 mg/g and 100 mg/g, relative to the total mass of said formulation.

In one embodiment, the formulation is characterised in that the concentration of macromolecular compound is between 2 mg/g and 75 mg/g, relative to the total mass of said formulation.

In one embodiment, the formulation is characterised in that the concentration of macromolecular compound is between 5 mg/g and 50 mg/g, relative to the total mass of said formulation.

In one embodiment, the formulation is characterised in that the concentration of macromolecular compound is between 10 mg/g and 40 mg/g, relative to the total mass of said formulation.

In one embodiment, the formulation is characterised in that the concentration of macromolecular compound is approximately 100 mg/g, relative to the total mass of said formulation.

In one embodiment, the formulation is characterised in that the concentration of macromolecular compound is approximately 80 mg/g, relative to the total mass of said formulation.

In one embodiment, the formulation is characterised in that the concentration of macromolecular compound is approximately 60 mg/g, relative to the total mass of said formulation.

In one embodiment, the formulation is characterised in that the concentration of macromolecular compound is approximately 40 mg/g, relative to the total mass of said formulation.

In one embodiment, the formulation is characterised in that the concentration of macromolecular compound is approximately 20 mg/g, relative to the total mass of said formulation.

In one embodiment, the formulation is characterised in that the concentration of macromolecular compound is approximately 10 mg/g, relative to the total mass of said formulation.

In one embodiment, the formulation is characterised in that it is injectable.

In one embodiment, the formulation is characterised in that it is sterile.

In one embodiment, the formulation is characterised in that it is injectable and sterile.

The formulations obtained based on compositions according to the invention have numerous applications.

Among the medical applications, we will cite for example injections to replace deficient biological fluids, for example in joints to replace synovial fluid, injection following surgery to avoid post-surgical adhesions, periurethral injections to treat incontinence and injections following presbyopia surgery. Among the aesthetic applications, we will cite for example injections for filling wrinkles, fine lines and skin defects or increasing volume, for example those of the lips, cheekbones, etc.

The targeted applications are more particularly the commonly used applications in the context of injectable viscoelastics and polysaccharides used or potentially usable in the following pathologies or treatments:
aesthetic injections on the face: to fill wrinkles, skin defects or volume (cheekbones, chin, lips);
volumizing injections at the body level: breast and buttock augmentation, G-spot augmentation, vaginoplasty, reconstruction of the vaginal lips, increase in penis size;
in joint surgery and dental surgery for filling periodontal pockets for example.
treatment of osteoarthritis, injection into the joint to replace or supplement deficient synovial fluid;
peri-urethral injection for the treatment of urinary incontinence due to sphincter insufficiency;
post-surgical injection to avoid peritoneal adhesions in particular;
injection following presbyopia surgery using laser scleral incisions;
injection into the vitreous cavity;
injection during cataract surgery;
injection for the treatment of cases of vaginal dryness;
injection into tissue spaces;
injection into the genitals.

More particularly, in cosmetic surgery, depending on its viscoelastic and persistence properties, the formulations obtained by the process which is the subject of the invention may be used:
for filling fine, medium or deep wrinkles, and be injected with fine diameter needles (27 Gauge for example);
as a volumizer with injection using needles of larger diameter, 22 to 26 Gauge for example, and longer (30 to 40 mm for example); in this case, its cohesive nature will guarantee its maintenance at the injection site.

These examples of use are in no way limiting, and the formulations obtained according to the process which is the subject of the invention are more widely intended for:
filling volumes;
generating spaces within certain tissues, thus promoting their optimal functioning;
replacing deficient physiological fluids.

The macromolecular compound according to the invention is not used in a mixture with a native polysaccharide in a formulation. In particular, the macromolecular compound according to the invention is not used in a mixture with hyaluronic acid or heparosan in a formulation.

In one embodiment, the macromolecular compound according to the invention is used in a mixture with at least one crosslinked polymer in a formulation.

In one embodiment, the macromolecular compound according to the invention is used in a mixture with at least one crosslinked polymer chosen from the group of crosslinked polysaccharides in a formulation.

In one embodiment, the macromolecular compound according to the invention is used in a mixture with at least one crosslinked polysaccharide chosen from the group consisting of hyaluronic acid, keratan, heparin, cellulose, cellulose derivatives, alginic acid, xanthan, carrageenan, chitosan, chondroitin, heparosan and their biologically acceptable salts, alone or in a mixture.

In one embodiment, the macromolecular compound according to the invention is used, alone or in a mixture with at least one other polymer, for the preparation of a crosslinked macromolecular compound.

In one embodiment, the macromolecular compound according to the invention is used in a mixture with at least one polysaccharide for the preparation of a crosslinked macromolecular compound.

In one embodiment, the polysaccharide used with the macromolecular compound according to the invention for the preparation of a crosslinked macromolecular compound is chosen from the group consisting of hyaluronic acid, keratan, heparin, cellulose, cellulose derivatives, alginic acid, xanthan, carrageenan, chitosan, chondroitin, heparosan and their biologically acceptable salts, alone or in a mixture, these polysaccharides being crosslinked or not crosslinked.

In one embodiment, the at least one polysaccharide used with the macromolecular compound according to the invention for the preparation of a crosslinked macromolecular compound is chosen from the group consisting of hyaluronic acid, heparosan and their biologically acceptable salts, alone or in a mixture, these polysaccharides being crosslinked or not crosslinked.

In one embodiment, the polysaccharide used with the macromolecular compound according to the invention for the preparation of a crosslinked macromolecular compound is a hyaluronic acid or a salt of hyaluronic acid.

In one embodiment, the polysaccharide used with the macromolecular compound according to the invention for the preparation of a crosslinked macromolecular compound is a mixture of hyaluronic acids or salts of hyaluronic acids.

In one embodiment, the polysaccharide used with the macromolecular compound according to the invention for the preparation of a crosslinked macromolecular compound is a hyaluronic acid or a salt of crosslinked hyaluronic acid.

In one embodiment, the polysaccharide used with the macromolecular compound according to the invention for the preparation of a crosslinked macromolecular compound is a heparosan or a salt of heparosan.

In one embodiment, the polysaccharide used with the macromolecular compound according to the invention for the preparation of a crosslinked macromolecular compound is a mixture of heparosan or heparosan salts.

In one embodiment, the polysaccharide used with the macromolecular compound according to the invention for the preparation of a crosslinked macromolecular compound is a heparosan or a salt of crosslinked heparosan.

In one embodiment, the macromolecular compound according to the invention is used for the preparation of at least one prodrug polymer.

In one embodiment, the macromolecular compound according to the invention is chemically modified by reaction with at least one active ingredient for the preparation of a prodrug polymer.

In one embodiment, the prodrug polymer obtained from the macromolecular compound according to the invention is used in the field of oncology,

### DESCRIPTION OF THE FIGURES

FIG. 1: Graph representing the evolution of the log (intrinsic viscosity—dL/g) curve as a function of log (molecular mass—Da) for each of the compositions studied in the examples.

FIG. 2: Diagrams representing a network formed during the crosslinking step, and its degradation during the steps of the process as claimed. Diagram A represents the intact network obtained in step d) of the process as claimed, the thick lines schematically represent the polysaccharide chains linked together, the thin lines schematically represent the divalent radicals L resulting from the crosslinker linked to the polysaccharide chains. Diagram B represents the breakage of the glycosidic bonds at different points of the previous network following the bond-breaking step e), represented by arrow 1, according to the claimed process. The thick lines schematically represent the broken polysaccharide chains, the dots symbolize the newly formed hydroxyl functions and the thin lines schematically represent the divalent L radicals resulting from the crosslinker. Finally, diagram C represents the mixed polymers of the macromolecular compound resulting from bond-breaking step e) according to the invention, arrow 2, unfolded in solution; where the thick lines schematically represent the broken polysaccharide chains, the dots symbolize the newly formed hydroxyl functions and the thin lines schematically represent the divalent L radicals resulting from the crosslinker.

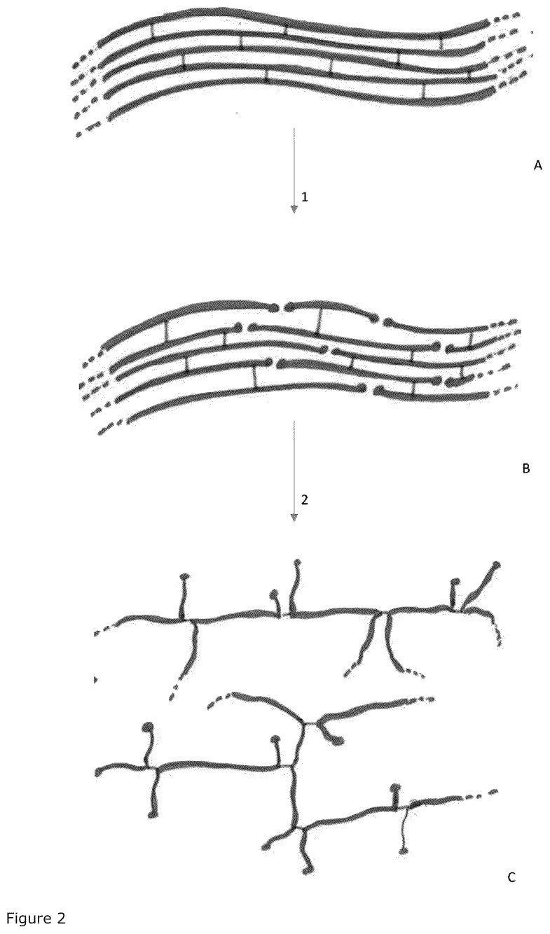

### EXAMPLES

As part of the examples, a number of parameters were measured.

Determination of the rheological parameters G′, G″ and Tan Δ: DHR-2 instruments TA apparatus. Cone type geometry with an angle of 2° and a diameter of 40 mm. Frequency sweep oscillation method, strain of 0.8% over a frequency range of 0.08 to 5 Hz.

Determination of the SEC: Viscotek GPCmax II apparatus equipped with a TDA305 detector (RI, RALS/LALS, Viscometer) and a Shodex OHpak SB-806 HQ+SB-805 HQ column. The measurements are carried out at 37° C. in the presence of PBS analysis buffer.

### Preparation of Compositions According to the Invention

### Example 1

Injectable grade sodium hyaluronate fibres (10.4 g) with a weight average molecular mass of 1 MDa are weighed in a container. An aqueous solution of 1% sodium hydroxide in water is added to hydrate the sodium hyaluronate fibres. The reaction medium is homogenized by alternating manual mechanical stirring and rest for 50 minutes. 0.43 g of BDDE is added. The reaction medium comprising 10.4 g of sodium hyaluronate fibres, 55.7 g of sodium hydroxide and 0.43 g of BDDE is again homogenized by manual mechanical stirring, then is placed in a previously thermostatically controlled water bath at 2° C. for 24 hours.

At the end of the crosslinking, the reaction medium is neutralized by adding 1 N HCl and phosphate buffer with mechanical stirring. The gel obtained is then dialyzed against phosphate buffer until a hyaluronic acid concentration of 27.5 mg/g is obtained. The phosphate buffer is prepared by solubilizing 0.23 g of NaH₂PO₄.2H₂O, 1.12 g of Na₂HPO₄, 42.5 g of NaOH in water for injection (EPPI) added in sufficient quantity to obtain 5 L of buffer solution. After mechanical homogenisation and debubbling, the gel obtained, whose Tan Δ at 1 Hz is 0.54, cannot be filtered on a membrane with a porosity of 0.22 μm; it is dispensed into 1 ml glass syringes and processed by steam sterilization at 127° C. with an F0 of 50 minutes. After this heat treatment, the syringes contain a solution of macromolecular compound. A sample is taken from a syringe, and a filterability test on a membrane with a porosity of 0.22 μm is carried out with this sample. The filterability test is positive: the macromolecular compound solution can be filtered on a membrane with a porosity of 0.22 μm.

### Example 2

A composition according to the invention is prepared by a process similar to that described in Example 1 but after addition of BDDE and homogenization, the reaction medium is immediately placed in a water bath previously thermostated at 9° C. for 3 hours at this temperature. As described in Example 1, before the heat treatment the gel, whose Tan Δ at 1 Hz is 0.68, cannot be filtered on a membrane with a porosity of 0.22 μm; it is dispensed into syringes. After heat treatment, the syringes contain a macromolecular compound solution at 27.5 mg/g. A sample is taken from a syringe, and a filterability test on a membrane with a porosity of 0.22 μm is carried out with this sample. The filterability test is positive: the macromolecular compound solution can be filtered on a membrane with a porosity of 0.22 μm.

### Example of Reference 3

Injectable grade sodium hyaluronate fibres (10.4 g) with a weight average molecular mass of 1 MDa are weighed in a container. An aqueous solution of 1% sodium hydroxide in water is added to hydrate the sodium hyaluronate fibres. The reaction medium is homogenized by alternating manual mechanical stirring and rest for 50 minutes. Phosphate buffer to replace BDDE is added. The phosphate buffer is prepared by solubilizing 0.23 g of NaH₂PO₄.2H₂O, 1.12 g of Na₂HPO₄, 42.5 g of NaOH in water for injection (EPPI) added in sufficient quantity to obtain 5 L of buffer solution. The reaction medium comprising 2.6 g of sodium hyaluronate fibres, 13.9 g of sodium hydroxide and 0.11 g of buffer and again homogenized by manual mechanical stirring, is then placed in a previously thermostated water bath at 9° C. for 3 hours.

At the end of these 3 hours of stirring, the reaction medium is neutralized by adding 1 N HCl and phosphate buffer with mechanical stirring. The gel obtained is then dialyzed against phosphate buffer until a hyaluronic acid concentration of 27.5 mg/g is obtained. After mechanical homogenisation and debubbling, the gel obtained, whose Tan Δ at 1 Hz is 1.59, can be filtered on a membrane with a porosity of 0.22 μm; it is dispensed into 1 mL glass syringes and processed by steam sterilization at 127° C. with an F0 of 50 minutes. After this heat treatment, the syringes contain a solution of macromolecular compound, A sample is taken from a syringe, and a filterability test on a membrane with a porosity of 0.22 μm is carried out with this sample. The filterability test is positive: the macromolecular compound solution can be filtered on a membrane with a porosity of 0.22 μm.

### Properties of the Compositions According to the Invention

The determined rheological properties of the compositions according to the invention are given in Table 1 below.

TABLE 1

| | | | Example of |
|---|---|---|---|
| | Example 1 | Example 2 | reference 3 |
| Polysaccharide | Hyaluronic | Hyaluronic | Hyaluronic |
| | acid | acid | acid |
| G′ (Pa) at 1 Hz of | 86 | 68 | 44 |
| the solution | | | |
| Tan Δ at 1 Hz of | 1.00 | 1.16. | 2.07 |
| the solution | | | |
| Filterability on | YES | YES | YES |
| membrane with | | | |
| porosity 0.22 μm | | | |
| at 20 mg/g | | | |

The compositions according to the invention are filterable on a membrane with a porosity of 0.22 μm and have a Tan Δ greater than or equal to 1.00.

### Hydrodynamic Behaviour of the Compositions According to the Invention in Comparison with the Reference Composition 3

The compositions were subjected to chromatographic analysis by steric exclusion in order to determine the coefficients of the Mark-Houwink relationship which is written:

[η]=K.M^{a}

[η] being the intrinsic viscosity.
M being the viscometric average molar mass.
This linear relationship makes it easy to determine the values of the coefficients a and K.
If we represent log([η]) as a function of log(M), we obtain a line of slope a and a Y-intercept log(K).

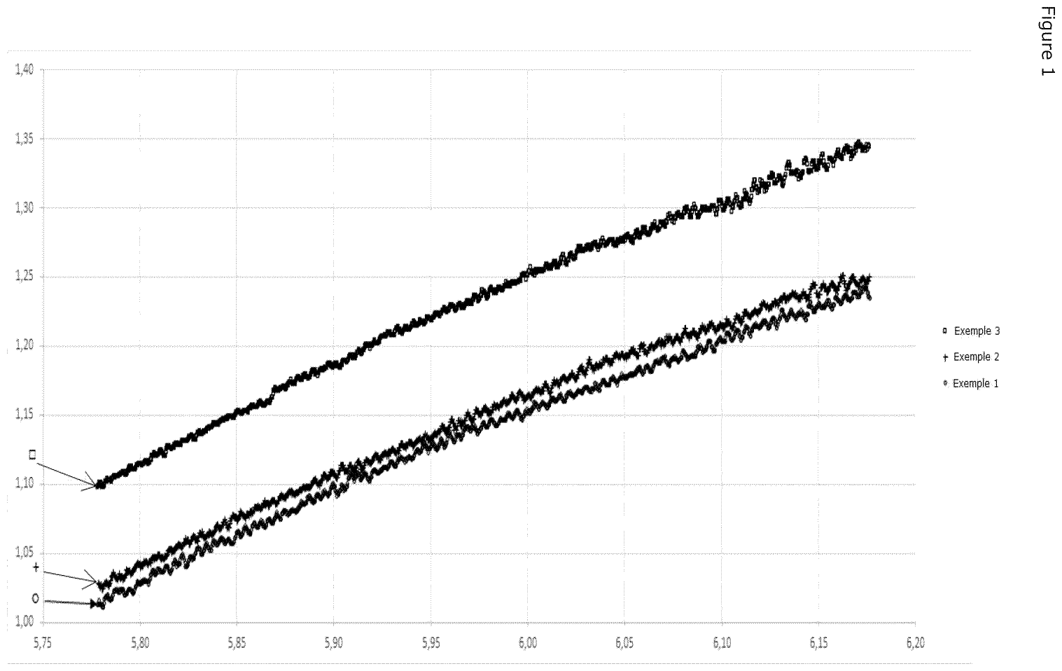

In FIG. 1, for each of the compositions the curve log(intrinsic viscosity—dL/g) as a function of log(molecular mass—Da) is plotted and the linear correlation is calculated.

The measured and calculated values are gathered in Table 2 below.

TABLE 2

| | | | | Coefficient “a” of | [η] Intrinsic |
|---|---|---|---|---|---|
| | | | | the Mark | viscosity for |
| | | Linear regression | Correlation | Houwink | Molecular mass = |
| Gel tested | Curves | equation | coefficient | relationship | 1,000 kDa |
| Example 1 | ∘ | Y = 0.564x − 2.238 | R² = 0.9941 | a = 0.56 | 14.1 dL/g |
| Example 2 | + | Y = 0.567x − 2.244 | R² = 0.9947 | a = 0.57 | 14.5 dL/g |
| Example of | □ | Y = 0.615x − 2.445 | R² = 0.9938 | a = 0.62 | 17.8 dL/g |
| reference 3 | | | | | |

It is observed that the hyaluronic acids of the compositions according to the invention have an intrinsic viscosity and a Mark Houwink coefficient similar to those of the hyaluronic acid of the composition of reference example 3 and are therefore no longer crosslinked.

Nevertheless, they present a G′ and a Tan Δ very different, suitable for the applications mentioned above.

### Example 4

Injectable grade sodium hyaluronate fibres (12.5 g) with a weight average molecular mass of 1 MDa are weighed into a container. An aqueous solution of 1% sodium hydroxide in water is added to hydrate the sodium hyaluronate fibres, The reaction medium is homogenized by alternating manual mechanical stirring and rest for 50 minutes. 0.56 g of BDDE is added. The reaction medium comprising 12.5 g of sodium hyaluronate fibres, 74.4 g of sodium hydroxide and 0.56 g of BDDE is again homogenized by manual mechanical stirring, then is placed in a previously thermostatically controlled water bath at 8° C. for 3 hours.

At the end of the crosslinking, the reaction medium is neutralized by adding 1N HCl and phosphate buffer with mechanical stirring. The phosphate buffer is prepared by solubilizing 5.40 g of NaH₂PO₄.2H₂O, 67.60 g of Na₂HPO₄, 240 g of NaOH and 4200 g of mannitol in water for injection (EPPI), added in sufficient quantity to obtain 120 L of buffer solution. The gel obtained is then dialyzed against this phosphate buffer containing 35 g/L of mannitol until a hyaluronic acid concentration of 27.5 mg/g is obtained.

The gel thus obtained cannot be filtered on a membrane with a porosity of 0.22 μm.

The gel, whose Tan Δ at 1 Hz is 0.70, is then divided into aliquots.

The first aliquot of 10.0 g of gel is acidified with 350 μL of 1N HCl to obtain a pH of 3. It degrades for 32 hours at 50° C. This gel is then neutralized with 1N NaOH to pH 7.3. Its rheological properties are measured.

A second aliquot of gel is exposed to radiation emitted by a 370 nm UV lamp with a power of 310 W for 397 hours. Once the exposure is finished, the rheological properties of the gel obtained are measured.

A third aliquot of 10.0 g of gel is thermostated at 37° C. in a syringe. A second syringe containing 100 μL of a hyaluronidase solution with a strength of 1750 units is thermostatically controlled at 37° C. The gel is mixed with hyaluronidase by connecting the 2 syringes via a double Luer connector then homogenized by going back and forth from syringe to syringe for 30 seconds. The mixture is then placed for 45 minutes at 37° C. At the end of this enzymatic degradation, the degradation is stopped by immersion for 4 minutes in a bath at 100° C. then brought back to 25° C. using an ice bath and its rheological properties measured.

All the results of the rheological measurements are brought together in Table 3 below.

TABLE 3

| Method of | Acid | UV | Enzymatic |
|---|---|---|---|
| degradation | degradation | degradation | degradation |
| G′ (Pa) at 1 Hz of | 30 | 39 | 28 |
| the solution | | | |
| Tan Delta at 1 Hz | 1.61 | 1.44 | 1.51 |
| of the solution | | | |

It is observed that whatever the degradation method applied, the Tan Δ (Tn δ) at 1 Hz of the compounds obtained is strictly greater than 1.00 (Tan Δ (Tn δ)>1.00).

The different aliquots obtained following the different degradation methods can all be filtered on membranes with a porosity of 0.22 μm.

### Example 5

A composition according to the invention is prepared by a process similar to that described in Example 4 until crosslinking. At the end of crosslinking, the reaction medium is neutralized by adding 1N HCl and phosphate buffer with mechanical stirring. The phosphate buffer is prepared by solubilizing 0.23 g of NaH₂PO₄.2H₂O, 1.12 g of Na₂HPO₄, 42.5 g of NaOH in water for injection (EPPI) added in sufficient quantity to obtain 5 L of buffer solution. The gel obtained is then dialyzed against phosphate buffer until a hyaluronic acid concentration of 20.0 mg/g is obtained.

The gel thus obtained cannot be filtered on a membrane with a porosity of 0.22 μm.

The gel, whose Tan Δ at 1 Hz is 0.79, is then divided into aliquots.

The first aliquot of 10.0 g of gel is placed in contact with a solution containing 37.5 μl of an aqueous solution of iron sulphate heptahydrate at a concentration of 15 mg/g and 75 μL of hydrogen peroxide at 30% by volume and homogenized by 60 syringe/syringe round trips in order to achieve radical degradation following the Fenton reaction. After 4 hours 20 minutes of contact, the gel is purified by dialysis against phosphate buffer. At the end of 2 dialysis baths lasting a total of 41 hours 30 minutes, its rheological properties are measured.

The second aliquot of 10.0 g of gel is acidified with 250 μL of 1N HCl to obtain a pH of 3. It is degraded for 14 hours 19 minutes at 50° C. This gel is then neutralized with 1N NaOH to pH 7.3. Its rheological properties are measured.

A third gel aliquot of 10.0 g of gel is thermostated at 37° C. in a syringe. A second syringe containing 50 μL of a hyaluronidase solution with a strength of 1750 units is thermostated at 37° C. The gel is mixed with hyaluronidase by connecting the 2 syringes via a double Luer connector then homogenized by going back and forth from syringe to syringe for 30 seconds. The mixture is then placed for 25 minutes at 37° C. At the end of this enzymatic degradation, the degradation is stopped by immersion for 4 minutes in a bath at 100° C. then brought back to 25° C. using an ice bath and its rheological properties measured.

All the results of the rheological measurements are brought together in Table 4 below.

TABLE 4

| Degradation | Radical | Acid | Enzymatic |
|---|---|---|---|
| method | degradation | degradation | degradation |
| G′ (Pa) at 1 Hz of | 28 | 31 | 27 |
| the solution | | | |
| Tan Delta at 1 Hz | 1.10 | 1.19 | 1.30 |
| of the solution | | | |

It is observed that whatever the degradation method applied, the Tan Δ (Tn δ) at 1 Hz of the compounds obtained is strictly greater than 1.00 (Tan Δ (Tn δ)>1.00).

The different aliquots obtained following the different degradation methods can all be filtered on membranes with a porosity of 0.22 μm.

1-14. (canceled)

15. A formulation, comprising a composition, in the form of an aqueous solution, filterable on a membrane of porosity 0.22 μm comprising at least one macromolecular compound consisting of sequences of identical or different polysaccharides linked together by a divalent radical, wherein the concentration of macromolecular compound is between 2 mg/g and 200 mg/g, relative to the total mass of the formulation.

16. The formulation according to claim 15, wherein the macromolecular compound is chosen from the compounds of general formula I: 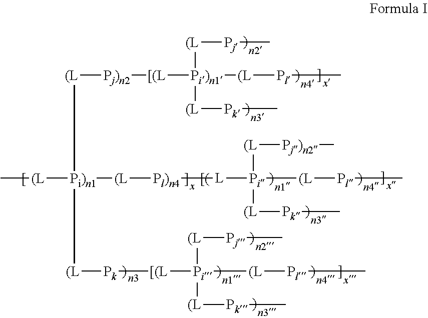
wherein:
Pᵢ, P_{i′}, P_{i″}, P_{i″′}, Pⱼ, P_{j′}, P_{j″}, P_{j″′}, Pₖ, P_{k′}, P_{k″}, P_{k″′}, Pₗ, P_{l′}, P_{l″} and P_{l″′} are identical or different polysaccharides,
n1, n1′, n1″, n1″′, n2, n2′, n2″, n2″′, n3, n3′, n3″, n3″′, n4, n4′, n4″ and n4″′ are integers between 0 and 2000 and at least one of n1, n1′, n1″, n1″′, n2, n2′, n2″, n2″′, n3, n3′, n3″, n3″′, n4, n4′, n4″ and n4″′ is greater than or equal to 2,
x, x′, x″ and x″′ are integers greater than or equal to 0,
L is a divalent radical resulting from a reaction between a crosslinking agent and two reactive functions, each reactive function being carried by two distinct polysaccharide chains,
sequences of polysaccharides do not form cyclic structures.

17. The formulation according to claim 15, wherein the macromolecular compound consists of a sequence of polysaccharides chosen from the group consisting of hyaluronic acid, keratan, heparin, cellulose, cellulose derivatives, alginic acid, xanthan, carrageenan, chitosan, chondroitin, heparosan, and their biologically acceptable salts, alone or in a mixture.

18. A macromolecular compound of general formula I: 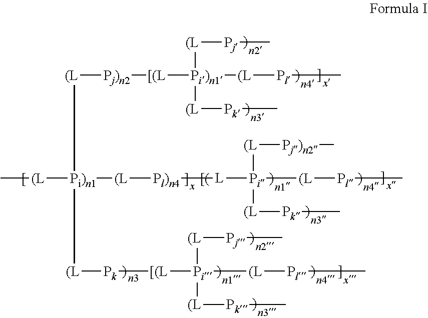
wherein:
Pᵢ, P_{i′}, P_{i″}, P_{i″′}, Pⱼ, P_{j′}, P_{j″}, P_{j″′}, Pₖ, P_{k′}, P_{k″}, P_{k″′}, Pₗ, P_{l′}, P_{l″} and P_{l″′} are identical or different polysaccharides,
n1, n1′, n1″, n1″′, n2, n2′, n2″, n2″′, n3, n3′, n3″, n3″′, n4, n4′, n4″ and n4″′ are integers between 0 and 2000 and at least one of n1, n1′, n1″, n1″′, n2, n2′, n2″, n2″′, n3, n3′, n3″, n3″′, n4, n4′, n4″ and n4″′ is greater than or equal to 2,
x, x′, x″ and x″′ are integers greater than or equal to 0,
L is a divalent radical resulting from a reaction between a crosslinking agent and two reactive functions, each reactive function being carried by two distinct polysaccharide chains,
sequences of polysaccharides do not form cyclic structures.

19. A process for preparing a composition, in the form of an aqueous solution, filterable on a membrane with a porosity of 0.22 μm comprising at least one macromolecular compound consisting of sequences of identical or different polysaccharides linked together by a divalent radical, comprising at least the following steps:
a) providing at least one polysaccharide
b) providing at least one crosslinking agent
c) carrying out one or more crosslinking step(s) in the presence of the polysaccharide and the crosslinking agent and a step of dilution of the crosslinked polysaccharide is carried out to obtain a concentration of crosslinked polysaccharide of between 2 mg/g and 200 mg/g, relative to the total mass of the compositions
d) obtaining a crosslinked polysaccharide
e) implementing one or more step(s) of breaking glycosidic bonds
f) obtaining a solution of macromolecular compound
g) filtering the macromolecular compound solution through a membrane with a porosity of 0.22 μm.

20. The process according to claim 19, wherein the crosslinking agent is chosen from the group consisting of bis-epoxides, trimetaphosphates, diamines, dialkoxyamines and dihydrazides.

21. A method according to claim 19, wherein step e) of breaking glycosidic bonds is carried out by means of chemical treatment.

22. The method according to claim 19, wherein step e) of breaking glycosidic bonds is carried out by means of heat treatment.

23. The method according to claim 19, wherein step e) of breaking glycosidic bonds is carried out by means of radiation treatment.

24. The process according to claim 19, wherein step e) of breaking glycosidic bonds is carried out by means of enzymatic treatment.

25. The method according to claim 19, wherein step e) of breaking glycosidic bonds is carried out by means of high-pressure treatment.

26. A composition obtained according to claim 19, wherein Tan Δ (Tn δ)≥1.00 at 1 Hz.

27. The formulation according to claim 15, wherein it further comprises at least one active ingredient.

28. The formulation according to claim 27, wherein the active ingredient is chosen from the group consisting of local anaesthetics, antioxidants, anti-inflammatoi vitamins, amino acids, vasoconstrictors, vasodilators, anti-haemorrhagics or haemostatics, antimicrobials, glycosides and their derivatives, moisturizers or tissue regenerators, alone or in a mixture.
